SEQUENCE LISTING (7 sequences)

SEQ ID NO: 1 - DNA, 21 bp, artificial
  oligonucleotides
cctgcctgctgtgcctgatgg

SEQ ID NO: 2 - DNA, 20 bp, artificial
  oligonucleotides
gctgacgtcaaagccccctc

SEQ ID NO: 3 - DNA, 77 bp, artificial
  oligonucleotides
ccatcaggcacagcaggcaggggtgggggatgtaaggaggggaaggtgggggacccagagggggctttgacgtcagc

SEQ ID NO: 4 - DNA, 77 bp, artificial
  oligonucleotides
ccatcaggcacagcaggcacatctgggggatgtaaggaggggaaggtgggggacccagagggggctttgacgtcagc

SEQ ID NO: 5 - DNA, 77 bp, artificial
  oligonucleotides
ccatcaggcacagcaggcaggggtgggggatgtaaggaggggaaggtgggggacccagagcatcctttgacgtcagc

SEQ ID NO: 6 - DNA, 20 bp, artificial
  oligonucleotides
gcacgagcccctggtccccg

SEQ ID NO: 7 - DNA, 20 bp, artificial
  oligonucleotides
  misc_feature (1)..(20): n is a, c, g, or t
nnnnnnnnnnnnnnnnnnnn

(19) United States
(12) Patent Application Publication
(10) Pub. No.: US 2024/0294919 A1
(43) Pub. Date: Sep. 5, 2024

(54) Modulating Human Tyrosine Hydroxylase Expression Through Control of Specific G-Quadruplex Formation

(71) Applicant: KENT STATE UNIVERSITY, Kent, OH (US)

(72) Inventors: Soumitra BASU, Hudson, OH (US); Nathan BEALS, Streetsboro, OH (US); Mohamed M. FARHATH, Dehiwala (LK); Prakash KHAREL, Jamaica Plain, MA (US)

(21) Appl. No.: 18/563,140

(22) PCT Filed: May 25, 2022
(86) PCT No.: PCT/US2022/030904

§ 371 Date: Nov. 21, 2023

Related U.S. Application Data
(60) Provisional application No. 63/192,781, filed on May 25, 2021.

(51) Int. Cl. C12N 15/113; A61K 47/69; A61P 25/16

(52) U.S. Cl. CPC C12N 15/1137; A61K 47/6923; A61K 47/6929; A61P 25/16; C12N 2310/151; C12N 2310/3513; C12N 2320/32; C12Y 114/16002

(57) ABSTRACT

Novel DNA molecule-based compositions and methods for delivery to target cells provide modulation of target G-quadruplex formation in the tyrosine hydroxylase (TH) promoter and allow for enhancement or repressing of specific G-quadruplexes that in turn regulate tyrosine hydroxylase transcription and catecholamine production, specifically dopamine. Use of the DNA-molecule-based compositions in treatment of neurological diseases and disorders is facilitated through a nanoparticle-based delivery system.

This invention was made with government support under Contract No. R15GM096285 awarded by the National Institutes of Health. The Government has certain rights in the invention.

### INCORPORATED BY REFERENCE

The Sequence Listing under document KENTBGPROVPCT_ST25.txt, created 05/25/2022 with 2,000 bytes is incorporated by reference.

### FIELD OF THE INVENTION

The present invention is directed to compositions and methods for modulating endogenous TH gene expression and, thus, dopamine production, by targeted switching of G-Quadruplexes (GQs) within the TH promoter, specifically 5′GQ and 3′GQ, to their active state using rationally designed DNA GQ Clips (5′GQ and 3′GQ Clips). The present invention is also directed to a targeted nanoparticle delivery system synthesized to effectively deliver the 5′GQ Clip and 3′GQ Clip in vivo. The invention provides an improved approach for controlling dopamine production in a multitude of neurological disorders including without limitation Parkinson's Disease (PD).

### BACKGROUND OF THE INVENTION

Catecholamines (CA) play important roles in brain functions, such as attention, memory, cognition, and emotion, making regulation of these biological molecules important in modulating neurological functions and disorders. The downstream products of the CA biosynthesis pathway, namely dopamine, epinephrine and norepinephrine, are vital as hormones and neurotransmitters within the central and peripheral nervous systems.

Tyrosine hydroxylase (TH) is a member of pterin-dependent monooxygenases which catalyze the rate-determining step of catecholamine (CA) biosynthesis. Specifically, TH catalyzes the hydroxylation of L-tyrosine to L-3,4-dihydroxyphenylalanine (L-DOPA) and is highly conserved.

Dopaminergic dysfunction in humans is associated with a variety of health conditions, including Parkinson's disease (PD), post-traumatic stress disorder (PTSD), schizophrenia, depression, drug addiction and attention deficient disorder (ADD). Within PD, several pathological events have been linked to dopamine dysfunction, such as elevated reactive oxygen species (ROS) through metal dysregulation, Lewy body formation, and transcriptional regulation of TH. The critical role of TH in dopamine biosynthesis makes transcriptional regulation of TH expression an important molecular target for controlling dopamine synthesis to assist in the treatment of several neurological disorders including PD.

Previously, it has been demonstrated that a Guanine-rich (G-rich) region (segment) within the TH promoter (main regulatory portion of a gene) plays an instrumental role in controlling the expression of the downstream gene. G-rich nucleic acid sequences having four G-stretches of two or more consecutive Gs can rearrange themselves into square planar structures called G-quartets. In the presence of monovalent cations, such as potassium and sodium, G-quartets can stack on top of each other to form a non-canonical secondary structure called a G-quadruplex (GQ). In many instances, potential GQ-forming sequences are located within important biological regions of the genome, such as the promoter regions and recombination sites. GQs within the promoter regions of many genes such as PDGF-A, VEGF, c-MYC, KRAS, and BCL2, act as a transcriptional repressor. Thus, the GQs in the gene promoter have been the obvious targets for small-molecule ligands that can control the stability of GQs to modulate transcription.

However, small molecule targeting has yielded mixed results because of limited capability to alter the stability of GQs and poor selectivity in the presence of other GQ targets. To overcome the paucity of specific GQ-recognizing ligands, nucleic acid-based therapeutics have been rationally developed (engineered) to precisely target the desired GQ regions of the TH promoter to modulate TH expression and, hence, to control the dopamine synthesis in neurons.

It has also been reported that a 45 nucleotide G-rich sequence (TH49) within the 3′-end of human TH promoter adopts two different sets of G-quadruplex (GQ) structures (5′GQ and 3′GQ, FIG. 1), where the 5′GQ structure enhances TH transcription and the 3′GQ structure represses it.

To exploit this unique feature of the GQ forming region to modulate dopamine level, a novel DNA molecule-based strategy has been developed, which can regulate target GQ formation. The newly designed (and engineered) GQ targeting DNA Clips (GQ Clips) are complementary to a segment of the TH49 sequence within the TH promoter as illustrated in FIG. 1. Binding of the engineered GQ Clip to the targeted G-stretches within TH49 blocks the formation of a particular GQ in the TH promoter and in turn, favors the formation of the other GQ.

The present invention is premised on the need to develop rational therapeutic design approaches for targeting and modulating the formation of specific GQs, specifically certain GQs within the endogenous human TH promoter. Previous strategies have been developed to target specific quadruplexes that take advantage of using antisense nucleic acid sequences to target nearby regions to achieve single GQ targeting precision. By way of example, Tassinari, et al. recently developed a naphthalene diimide-peptide nucleic acid (PNA) complex that was able to distinguish specific GQs while also using the aromatic naphthalene to improve the stability of the target GQ. The small molecule-PNA conjugate was able to recognize specific GQs of choice within the HIV-LTR based on the sequence of the antisense PNA as a new GQ targeting therapeutic approach. In another approach, a locked-nucleic acid therapeutic was designed that was able to modulate the equilibrium in the pre-miRNA 92b structure between a folded GQ and duplex products leading to an anticancer effect. In another approach, synthetic RNA-DNA GQs within the 5′ UTR of eIF-4E mRNA were induced as designed roadblocks to prevent protein production. However, these strategies, along with other nucleic acid therapeutic approaches discussed below, possess pharmacokinetic and other disadvantages, making a delivery approach essential for in vivo efficacious treatment.

The use of nucleic acid therapeutics has been a promising idea for the treatment of brain disorders; however, the underlying pitfalls make it challenging to accomplish therapeutic success. Some of the obstacles include off-target toxicities, limited localization at diseased sites due to poor serum stability, passage through the blood brain barrier, and lack of cellular recognition. To date, several nanoparticle compositions have established the value of material complexes for the treatment of neurodegenerative diseases. Two small molecule nanoparticle delivery approaches for brain disorder treatment consisted of a poly(n-butylcyanoacrylate) and polysorbate 80 nanoparticle for treating Alzheimer's resulting in a 4-fold increase in rivastigmine uptake, while the other used a peptide targeted PLGA-PEG nanoparticle for the improved delivery of odorranalectin for the treatment of PD. Additionally, a PEI nanoparticle was complexed with α-synuclein siRNA through charge-charge interactions to create longer circulation times and increased stability of the siRNA cargo. The nanoparticles were able to localize in dopaminergic neurons and reduce protein expression in a dose-dependent manner.

While the delivery of the DNA molecules to neuronal cells is difficult, targeted delivery of DNA sequences to specific cells within the animal brain is undoubtedly even more challenging. Nanoparticle drug delivery has been used to overcome the limitations or disadvantages that individual therapeutics have and are useful to improve upon drug retention, protection, and circulation. Specific examples are gold nanoparticle and DNA nanoconjugates, referred to as spherical nucleic acids (SNAs), that have displayed success in protecting against DNA degradation and prolonging bioavailability for the treatment of various cancers. The addition of targeting moieties allows for specific cellular uptake, which is highly important for disease treatment, and an adjacent cellular entry pathway that can circumvent poor uptake of large, highly negatively charged DNA molecules.

There remains a need for alternative design DNA-based molecules to target and modulate the formation of specific GQ's. There also remains a need for an effective delivery system for DNA-based molecules without the disadvantages of prior approaches.

To harness therapeutic advantage in terms of both targeting and efficient delivery, a nanoparticle delivery system has been developed and synthesized to deliver the 5′GQ Clip to dopaminergic neurons via targeting with a TrkB peptide aptamer, which exhibited a significantly improved response in cellulo compared to delivery of a 5′GQ Clip only. Additionally, using a transgenic rat model in which the human TH promoter drives the expression of the reporter gene GFP (green fluorescent protein), enhanced GFP production in the rat brain was achieved. Through modulating TH promoter activity, it is believed this nucleic acid drug delivery system is an effective mechanism for controlling dopamine production, which eventually can help to treat various neurological disorders, especially Parkinson's, while potentially reducing the side effects commonly seen with current treatments.

It is an object of the invention to provide engineered GQ-targeting DNA Clips (GQ Clips) that are complementary to and capable of targeting a segment of the TH 49 sequence within the TH promoter to modulate formation of specific GQs within the segment.

It is another object of the invention to provide a nanoparticle-based delivery system to facilitate the delivery of the GQ-targeting DNA Clips (GQ Clips) to the target segment of the TH 49 sequence.

Still another object of the invention is a method for regulating catecholamine production to modulate neurological functions and treat neurological disorders.

Yet another object of the invention is a method for increasing dopamine production through the administration of GQ-targeting DNA Clips to a human subject.

A further object of the invention is to provide a method for treating Parkinson's disease through the administration of GQ-targeting DNA Clips to a human subject and avoid the side effects commonly seen with current conventional treatments.

Other objects of the invention will be evident to one skilled in the art based on the disclosure herein.

### SUMMARY OF THE INVENTION

The invention is directed to novel DNA molecule-based strategies to regulate target GQ formation. Specifically, the invention is directed to novel, engineered GQ-targeting DNA Clips (GQ Clips) that are complementary to a segment of the TH49 sequence in the TH promoter. The invention is also directed to a novel nanoparticle or DNA nanoconjugate delivery system comprising the novel GQ Clips to prolong bioavailability and protect against DNA degradation, thus improving drug retention, protection and circulation.

The invention is further directed to novel methods for modulating the TH promoter activity to control dopamine and other neurotransmitter production to facilitate treatment of neurological disorders, while avoiding the side effects common with current, traditional treatments.

In one embodiment, the invention is an engineered G-quadruplex (GQ)-targeting DNA Clip that is complementary to and targets a segment of human tyrosine hydroxylase promoter.

In another embodiment, the invention is an exogenous, engineered 5′GQ DNA Clip or a 3′GQ DNA Clip, wherein the 5′GQ DNA Clip enhances tyrosine hydroxylase transcription and the 3′GQ DNA Clip represses tyrosine hydroxylase transcription.

Another embodiment of the invention is a nanoparticle drug delivery system that delivers the G-quadruplex (GQ)-targeting Clips to a human subject through targeting with a Trk-B peptide aptamer.

Yet another embodiment of the invention is a method for controlling dopamine production in a human subject by providing an engineered G-quadruplex (GQ)-targeting DNA Clip to a human subject via a nanoparticle drug delivery system that delivers the clip to a dopaminergic neuron via targeting with a TrkB peptide aptamer.

Still another embodiment of the invention is a method of treating Parkinson's disease comprising the step of administering the engineered G-Quadruplex targeting DNA Clip to a human subject.

Other embodiments will be evident to one skilled in the art based upon the disclosure herein.

### BRIEF DESCRIPTION OF THE FIGURES

FIG. 1 is a schematic representation of the position of TH49 within the TH promoter and reflects a strategy for blocking a specific GQ formation in the TH promoter using the inventive GQ Clips.

FIGS. 2A, 2B, 2C and 2D show that the inventive GQ Clips bind to TH49 to elicit structural changes. FIGS. 2A and 2B show binding to TH 49 in dose dependent fashion.

FIG. 2C shows that the 5′GQ Clip blocks formation of the 3′GQ structure. Native PAGE of TH49 GQ (1 μM) in the absence and presence of increasing concentrations of 5′GQ Clip or 3′GQ Clip. (0, 1, 10 and 50 μM). The 5′GQ Clip blocks the formation of 3′GQ structure. DMS structural mapping of wtTH49 in the presence of systematically increasing the concentration of 5′GQ Clip. Lane designations are as follows: Lane 1, DMS probing at 0 mM K⁺; Lane 2, DMS probing at 100 mM K⁺; Lane 3-5, DMS structural mapping in the presence of increasing concentrations of 3′GQ Clip at 100 mM K⁺. The TH49 sequence is shown on the left side and the G-stretches in the sequence and corresponding bands in the gel are labelled as VII (GGGG-3′). FIG. 2D shows that the 3′GQ Clip blocks the formation of the 5′GQ structure. DMS structural mapping was repeated with the 3′GQ DNA Clip. The TH49 sequence is shown on the left side and the G-stretches in the sequence and corresponding bands in the gel are labelled as I (5′-GGGG).

FIGS. 3A, 3B and 3C show that the inventive GQ Clips modulate tyrosine hydroxylase expression and dopamine production in dopaminergic neurons. FIG. 3A shows that the rationally designed GQ Clips modulate the endogenous TH transcription in human SH-SY5Y cells. Histograms representing the ratio of endogenous TH to GAPDH mRNA levels in SH-SY5Y cells were performed. The mRNA level in the cells treated with scramble Clip was set to 1, and mRNA from GQ Clip treated cells were normalized accordingly (data represent mean values±the standard error of the mean; n=9; ***=P<0.001 from a Student's t-test). FIG. 3B shows that GQ Clips modulate the endogenous TH protein expression. TH protein levels in SH-SY5Y cells were treated with 5′GQ Clip, 3′GQ Clip, and scrambled Clip as the control oligonucleotide. Western blot of the TH protein expression levels when treated with various oligonucleotides was performed. Histogram representing densitometry analysis of the western blot image was performed using Image J software. The TH bands were normalized with corresponding GAPDH bands to correct for experimental loading errors. FIG. 3C shows targeted control of endogenous dopamine production in SH-SY5Y cells by GQ Clips. Cellular dopamine levels in SH-SY5Y cells treated with 5′GQ Clip, 3′GQ Clip, and scrambled Clip sequences. The dopamine level of 2×10⁶ cells was measured using HPLC equipped with ECD. The histogram represents a quantitative analysis of the cellular dopamine level. Data represented as mean±SEM and the significance of the data was determined by t-test analysis. (***=p<0.001)

FIGS. 4A through 4F show synthesis of a 5′GQ Clip nanoparticle system for the targeted delivery of 5′GQ Clip. FIG. 4A shows a schematic of targeted clip nanoparticle for treatment of Dopaminergic neurons. FIG. 4B shows TEM of AuNP. The black bar represents 10 nm. FIGS. 4C and 4D show UV-Vis Spectroscopy used to determine the loading and stability of the 5′GQ Clip nanoparticle. The absorbance of DNA at 260 nm was measured to quantitate conjugation onto the nanoparticle complex, as after conjugation and purification the increase in intensity was used to measure loading efficiency (˜85%). The 5′GQ Clip nanoparticle was incubated in increasing concentrations of NaCl to assess the stability of the complex in salt. Through the addition of 500 mM NaCl, no redshift or broadening of the 515 nm AuNP specific peak was observed, which both resemble nanoparticle aggregation. FIGS. 4E and 4F show in vitro release of Clip DNA from 5′GQ Clip nanoparticle via increased glutathione level. Glutathione has an affinity for the gold surface (ligand-metal interactions between thiol and gold), replacing the thiolated DNA. The DNA was radiolabeled, and the higher band resembled bound DNA to the nanoparticle complex, while the lower band was released DNA after being incubated in 10 mM glutathione for various time points. The bands were quantified using Image J software and the data plotted.

FIGS. 5 A, 5B and 5C show that the 5′GQ Clip nanoparticle improves cellular efficacy. FIG. 5A shows neuronal uptake of 5′GQ Clip nanoparticle is dependent on the Trk-B aptamer for successful cellular recognition. SH-SY5Y cells were incubated with 270 nM free fluorescently labelled 5′GQ Clip, No aptamer 5′GQ Clip nanoparticle, or 5′GQ Clip nanoparticles (with TrkB). After 24 hours, the TrkB aptamer was shown to be essential for cellular uptake presumably through receptor-mediated endocytosis and eventually reaching the nucleus. Scalebar: 10 μM. FIGS. 5B and 5C show that the 5′GQ Clip nanoparticle dramatically increased the expression of TH. SH-SH5Y cells were treated with 270 nM 5′GQ Clip nanoparticle and 5′GQ Clip for 24 Hrs. Cellular RNA and protein were extracted and the TH expression was normalized to GAPDH showing the nanoparticle complex increased expression by 9-fold and 6-fold respectively. Data was represented as mean±SEM (**=p<0.01). FIGS. 5D and 5E show 5′GQ Clip nanoparticle treatment can improve TH expression in disease state cells. SH-SH5Y cells were treated with 270 nM 5′GQ Clip nanoparticle in the presence of 5 μM 60HDA for 24 hrs. Similar to A and B, RT-qPCR and Western blot were used to verify the nanoparticle complex can increase mRNA and protein expression of TH 3-fold in the presence of high oxidative stress mimicking PD disease state. Data represented as mean±SEM (**=p<0.01).

FIGS. 6A, 6B, 6C and 6D show that the 5′GQ Clip nanoparticle is taken up by neurons and increases the expression of GFP in TH-GFP transgenic rat model. FIG. 6A shows that Confocal fluorescent images display successful cellular recognition and uptake of the 5′GQ Clip nanoparticle complex in rat brains. Rats were treated with 10 μg of total 5′GQ Clip DNA (conjugated to the 5′GQ Clip nanoparticle). After 24 hours, cellular uptake in the substantia nigra of treated rats. FIG. 6B: Confocal fluorescent images showed successful upregulation of GFP through targeting of the TH promoter by the 5′GQ Clip nanoparticle complex in rat brains. Transgenic TH-GFP rats were treated with 10 μg 5′GQ Clip or scrambled nanoparticles. After 24 hours, GFP expression was assessed of the substantia nigra of treated rats. The activity of the TH promoter is shown to increase greatly in the presence of the 5′GQ Clip compared to the scramble sequence (or endogenous GFP expression). Scalebar: 100 μM. FIG. 6C shows that the 5′GQ Clip nanoparticle treatment in TH-GFP rats achieved a 5-fold increase in GFP mRNA expression. Cellular RNA was extracted, and RT-qPCR was used to quantify GFP mRNA concentrations compared to GAPDH. FIG. 6D shows that GFP protein expression is increased with 5′GQ Clip nanoparticle treatment in TH-GFP rats. Quantification of GFP within rats (n=3) of the substantia nigra shows an average of a 5-fold increase in fluorescence per cell. Data represented as mean±SEM (*=p<0.05).

FIG. S1 is a DART-MS analysis of the HPLC analyzed fraction that further confirms the presence of dopamine (m/z 154.066, an H+ adduct).

FIG. S2 shows migration differences between Tfr1 DNA aptamer conjugated to bifunctional 5K PEG (left) and free Tfr1 DNA aptamer (right).

FIG. S3 shows a schematic of TrkB peptide PEGylation.

FIG. S4 shows the quantification of TrkB peptide aptamer post HPLC purification.

FIG. S5 shows the mass spectrum of purified TrkB peptide aptamer.

FIG. S6 shows the mass spectrum of TrkB peptide aptamer conjugated with BM (PEG).

FIG. S7 shows that the PEGylated Tfr1 aptamer was conjugated to 5′GQ Clip nanoparticle as reflected by the migration of the radioactive band on an agarose gel.

FIG. S8 shows 5′GQ Clip nanoparticle migration in 1.5% agarose at varying DNA concentrations to observe DNA loading.

FIG. S9 shows that the 5′GQ Clip nanoparticle controls display limited tyrosine hydroxylase protein expression compared to untreated cells.

FIG. S10 shows that tyrosine hydroxylase mRNA increased as a function of dose-dependent 5′GQ Clip nanoparticle treatment of SH-SH5Y cells.

FIG. S11 shows increasing 5′GQ Clip nanoparticle concentrations that led to increasing tyrosine hydroxylase protein expression in the presence of neurotoxin.

FIG. S12 shows MTS of SH-SY5Y cells with varying concentrations of 60HDA with and without the presence of 5′GQ Clip nanoparticle, demonstrating that the 5′GQ Clip nanoparticle had a protective effect.

FIG. S13 shows increased GFP expression in rat substantia nigra with treatment of 5′GQ Clip nanoparticle.

Table S1 shows dynamic light scattering measurements of nanoparticle conjugates (DNA therapeutic, AuNP-PEG (pegylated), and 5′GQ Clip nanoparticle) measured at pH 6.5.

Table S2 shows zeta potential measurements in water performed for the 5′GQ Clip nanoparticle conjugates (DNA GQ Clip, AuNP-PEG, AuNP-DNA, and Clip Nanoparticle).

### DETAILED DESCRIPTION OF THE INVENTION

The invention is directed to novel DNA molecule-based strategies to regulate target GQ formation. Specifically, the invention is directed to designing and engineering novel GQ-targeting DNA Clips (GQ Clips) that are complementary to a segment of the TH49 sequence within the TH promoter. The invention is also directed to a novel nanoparticle or DNA nanoconjugate delivery system comprising the novel GQ Clips to prolong bioavailability and prevent DNA degradation and to improve drug retention, protection and circulation.

The invention is further directed to novel methods for modulating the TH promoter activity to control dopamine and other neurotransmitter production to facilitate treatment of neurological disorders, while avoiding the side effects common with current, traditional treatments.

For purposes of the invention, the following terms are defined.

“G” or “G-rich” means and incudes guanine and guanine-rich regions within the human TH promoter. G-rich nucleic acid sequences having four G-stretches of two or more consecutive guanines can rearrange themselves into square planar G-quartets. Quartets that stack on top of each other in the presence of monovalent cations, such as potassium and sodium, form a non-canonical secondary structure known as a G-quadruplex or GQ. “G-quadruplex” and “GQ” are used interchangeably herein.

“GQ Clips” or “GQ-targeting DNA Clips” or “G-quadruplex (GQ)-targeting DNA Clips” mean and include DNA molecules designed and engineered to specifically block the formation of either the 5′GQ or 3′GQ structures within the TH49 sequence of the TH promoter. “5′GQ Clip” and “3′GQ” Clip mean a DNA molecule complementary to the GQ structure within the TH49 sequence of the TH promoter.

“TH49” means a 45-nucleotide (nt) G-rich sequence within the 3′ end of the human TH promoter that adopts two different sets of GQ structures (5′GQ or 3′GQ). The 45-nucleotide (nt) sequence within the human TH promoter is also referred to as “wtTH49.”

GQ structures have attracted interest as therapeutic targets due to the diverse roles they play at different stages in gene regulation with profound implications in many human diseases, such as cancer, diabetes, neurodegeneration, and cardiovascular disease. The most common approach for targeting GQs has been the use of small molecule ligands with the intention to differentiate GQs from other secondary structures, including the non-targeted GQs, and alter their stability. Nevertheless, the main obstacles for using small molecules to target GQs in the cell have been their lack of specificity towards the targeted GQ and bioavailability.

The induction of selective GQ formation in the TH promoter (for example the 5′GQ) by small molecules is even more challenging due to the low level of structural variation between the 5′GQ and 3′GQ structures, making it difficult to target one over the other to modulate the increase or decrease of TH expression, respectively. The present invention is premised on the need to develop rational therapeutic design approaches for targeting a specific set of GQs within the endogenous human TH promoter. As discussed herein, other strategies have been developed; however, these strategies, along with other nucleic acid therapeutic approaches, possess pharmacokinetic disadvantages, making a delivery approach essential for in vivo efficacy and treatment. Obstacles encountered in use of nucleic acid therapeutics include toxicities, instability of the molecule resulting in poor localization at a disease site, passage through the blood-brain barrier, and lack of cellular recognition.

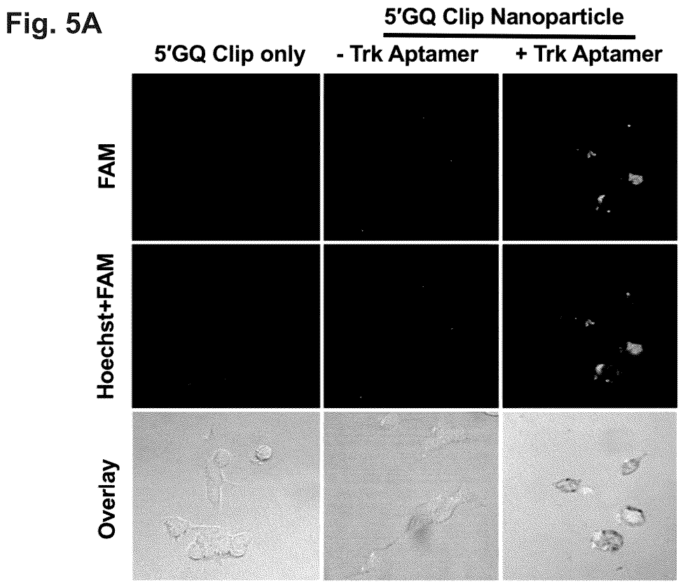

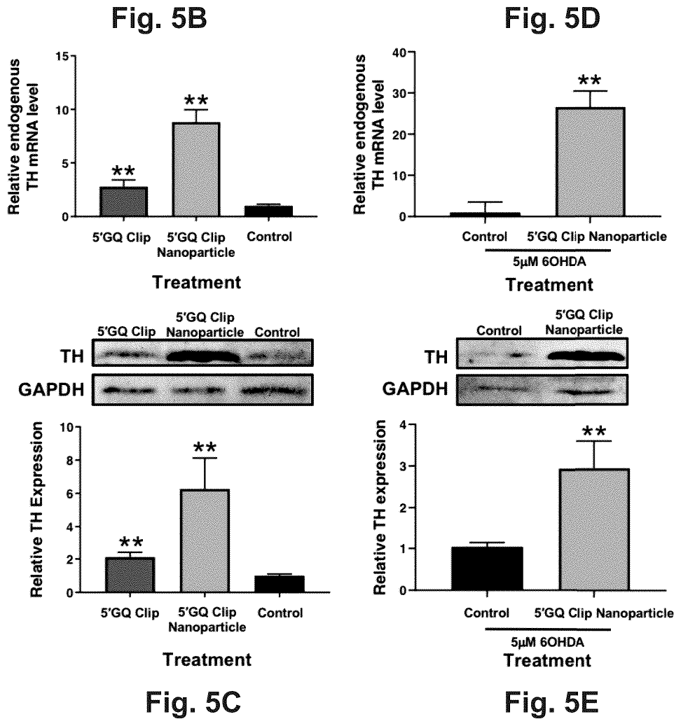

The present invention advances the ability to use nanomaterials for effective treatment of neurodegeneration by utilizing targeting ligands to actively focus on the dopaminergic neurons and using a polymer coating for stability and elongated circulation time. The addition of the aptamers has allowed the 5′GQ Clip nanoparticle to elicit better outcomes when compared to the 5′GQ Clip treatments alone as was evident from the RT-qPCR and WB data. Without wishing to be bound by theory, two major explanations or factors could potentially explain these results. First, the nanoparticle has inherent protection against DNA degradation through the PEG shell that presumably engulfs the DNA, allowing for limited accessibility to nucleases. The increased stability could potentially provide the 5′GQ Clip longer availability in cell studies. Second, targeting aptamers are known to enter the cell via receptor-mediated endocytosis, making cellular uptake more efficient as compared to DNA only. DNA molecules do not efficiently pass through the cell membrane due to high net negative charges and size, which is displayed by the minimal uptake of DNA alone observed in the Confocal microscopy experiments (FIG. 5A). Both of these factors are thought to contribute to a 4.5-fold increase in mRNA and a 3-fold increase in protein production in cellulo compared to the DNA treatment, further displaying the ability to increase in vivo GFP mRNA and protein production both by 5-fold in the TH-GFP transgenic rat model.

There are also a number of strategies that can be implemented to improve upon current nucleic acid drug delivery obstacles, such as endosomal escape, within the nanoparticle complex. Small molecules (chloroquine), pore-forming peptides, and fusogenic biomolecules have shown promise to induce endosomal escape as improvements to nanomaterial design, which may further improve the efficacy of the DNA GQ Clips. Together these highlight the beneficial pairing of nucleic acid therapeutics and nanotechnology that can be extended for a range of drug delivery applications in brain disorder treatment.

Parkinson's disease is one of the most common neurodegenerative diseases where treatment methods are centered around increased dopamine levels. But as with most neurological disorders, available treatment options are non-curative and can become less effective with the progression of the disease. L-DOPA therapy is the main treatment used currently for dopamine deficiency since replacement therapy with dopamine is not possible due to its inability to cross the brain capillary endothelial wall, which forms the blood brain barrier in vivo. Although L-DOPA replacement therapy has been the basis of dopamine deficiency for a long period, this treatment method has shown many side effects including difficulties in performing voluntary movements (dyskinesia), gastroesophageal reflux, vomiting and suppression of appetite. Most importantly, typically 4-6 years after starting treatment, patients will develop motor complications. These obstacles make finding new options to develop co-treatment or replacement of current L-DOPA therapy imperative. Monoamine oxidase B inhibitors (MAOB) have been studied as a possible pathway to further increase plasma dopamine levels in conjunction with L-DOPA treatment as a possibility to improve drug effectiveness but also possesses side effects. Zhang et al. reported an alternative approach to biologically replacing dopamine production using TH transvascular gene therapy with a transferrin antibody conjugated immunoliposomes. The gene therapy conjugate was able to specifically increase TH production in vivo, displaying the ability to improve upon motor symptoms in a 60HDA toxin model.

Because the sequence varies at the all-important G-rich region between the primate and rodent TH promoter sequences, a transgenic TH-GFP rat model was used to verify the ability of the 5′GQ Clip nanoparticle in vivo by targeting the human TH promoter. It is believed that this approach of delivering 5′ GQ Clips provides the capability to create a much higher level of precision when it comes to dopamine production compared to the current known approaches. It is also important to note that this strategy can not only increase dopamine production as observed in case of the 5′GQ Clip but also the ability to reduce dopamine production as achieved by using the 3′GQ Clip, which can be instrumental in several other dopamine related diseases, such as PTSD.

The present invention verifies that the targeting of the TH promoter region can systematically control dopamine production, which can be beneficial for various neurological diseases, especially when harnessed the dopamine enhancing role of the 5′GQ Clip, which can directly improve upon many disease treatments where increase in dopamine levels is necessary.

Based on previous concepts, a strategy was developed to modulate GQ function using nucleic acid therapeutics, wherein the strategy modulated the formation of two separate GQ structures within the same 49nt stretch of the human TH promoter shown to play an instrumental role in controlling TH expression and dopamine production. By pairing the nucleic acid therapeutics with a nanoparticle platform, the modulation of the human TH promoter activity in cellulo for both normal and 60HDA stressed human neuronal cells were improved. Increased TH activity in the in vivo TH-GFP transgenic rat model further supports the viability of the 5′GQ Clip nanoparticle as a proof of concept for neurological diseases, such as PD, where specifically further analysis is needed to observe how the increase in TH level affects the disease condition. The development of a humanized TH model in a rodent system will be vital to evaluate the potential effects of the TH modulation on motor symptoms in PD.

The invention is illustrated by the following non-limiting examples.

### EXAMPLES—MATERIALS AND METHODS

### DNA Oligonucleotide Preparation

All of the DNA oligonucleotides used in this study were purchased from Integrated DNA Technologies, Inc. Oligonucleotides were purified using 17% denaturing PAGE and were extracted via a crush and soak method by tumbling the gel slices at 4° C. in a solution of 300 mM NaCl, 10 mM Tris-HCl (pH 7.5), and 0.1 mM EDTA. Samples were concentrated with 2-butanol and ethanol precipitated with 3 volumes of ice-cold 100% ethanol. The salt was removed by washing the DNA pellets with ice-cold 70% ethanol.

Oligodeoxynucleotide sequences used in the study are set forth in Table 1 below. An asterisk (*) denotes phosphorothioate modification which renders the oligonucleotide exonuclease resistant.

TABLE 1

| Oligo name | Sequence |
|---|---|
| 3′GQ Clip | 5′ C*C*TGCCTGCTGTGCCTGAT*G*G |
| 5′GQ Clip | 5′ G*C*TGACGTCAAAGCCCCC*T*C |
| wtTH49 | 5′ CCATCAGGCACAGCAGGCAGGGGTGGGGGATGTAAGGAG |
| | GGGAAGGTGGGGGACCCA GAGGGGGCTTTGACGTCAGC |
| TH49 5′GQ mut | 5′ CCATCAGGCACAGCAGGCACATCTGGGGGATGTAAGGAGGGGA |
| | AGGTGGGGGACCCAGAGGGGGCTTTGACGTCAGC |
| TH49 3′GQ mut | 5′ CCATCAGGCACAGCAGGCAGGGGTGGGGGATGTAAGGAGGGGA |
| | AGGTGGGGGACCCAGAGCATCCTTTGACGTCAGC |
| Promotor control | 5′ G*C*ACGAGCCCCTGGTCCC*C*G |
| Scramble control | 5′ N*N*NNNNNNNNNNNNNNNN*N*N |

### Radiolabeling of DNA Oligonucleotides

The DNA sequences were 5′-end-radiolabeled by incubating with T4 polynucleotide kinase (NEB) and [γ-³²P]ATP (PerkinElmer) for 45 min. at 37° C. The radiolabeled DNA oligonucleotides were purified by 17% denaturing PAGE (polyacrylamide gel electrophoresis) and extracted from the gel via the crush and soak method.

### Native Gel Electrophoretic Mobility Shift Assay

The blocking of specific GQ formation was achieved by mixing of the TH49 template with an increasing amount of the corresponding GQ Clip (1:1, 1:10, 1:50) in 150 mM KCl, 10 mM Tris-HCl and 0.1 mM EDTA (pH 7.5). Then, the mixtures were heated to 95° C. for 10 min followed by slow cooling to room temperature over a 90 min. period. The complexes were resolved by 10% native polyacrylamide gel electrophoresis in Tris-borate-EDTA buffer supplemented with 150 mM KCl for 6 hrs. in a 4° C. cold room. The gel was exposed to a phosphorimager screen and then visualized by Typhoon Phosphorimager FLA 9500 (GE Healthcare, Life Sciences).

### Dimethyl Sulfate (DMS) Structure Mapping

Samples for DMS structure mapping were prepared by mixing the appropriate amount of GQ Clips with 1 μM unlabeled TH49 template, 10 mM Tris-HCl buffer (pH 7.4), 100,000 cpm 5′-end radiolabeled wtTH49 template, and 150 mM KCl or no KCl in a final volume of 30 μL. DNA structures were folded as described above. Then samples were treated with 1% DMS for 2 min. at room temperature before the methylation reactions were stopped by adding 300 μL of stop buffer (300 mM sodium acetate, 250 mg/mL sheared salmon sperm DNA, and 2 M P-mercaptoethanol). DNA samples were ethanol precipitated with 3 volumes of ice-cold 100% ethanol, and the DNA pellets were washed with 70% ethanol. The pellets were then dried in a vacuum centrifuge and then treated with 70 μL of freshly prepared 10% piperidine for 30 min. at 95° C. The cleaved products were resolved on a 12% denaturing polyacrylamide gel, and the dried gel was exposed to a phosphorimager screen and visualized on a Typhoon FLA 9500 Phosphorimager (GE Life Sciences).

### Cell Culture

The SH-SY5Y cells were cultured in Eagle's Minimum Essential Medium (EMEM)/F-12 (Corning) supplemented with 10% fetal bovine serum (FBS), 1% antibiotics streptomycin, penicillin, and amphotericin B, at 37° C. in 5% C02 in a humidified incubator. The cells were grown in 6-well plates with ˜500,000 cells per well and allowed to grow until they reached ˜80% confluency.

### Quantitative RT-PCR

Cells were treated for 24 hours with media containing Clip sequences, scrambled sequence, or nanoparticle complexes. The cells were then washed three times with full growth media and total cellular RNA was extracted from treated SH-SY5Y cells using a trizol reagent as per manufacturer's protocol. The cDNA was synthesized using qScript™ cDNA SuperMix (Quanta Biosciences). The glyceraldehyde 3-phosphate dehydrogenase (GAPDH) and TH mRNAs were subjected to RT-qPCR using a Perfecta® SYBR® Green Super Mix (Quanta Biosciences) on an Eppendorf Mastercycler® RealPlex2 in the presence of the appropriate set of primers. The relative mRNA levels were estimated by the comparative Ct method (Livak method).

For transfection of the GQ DNA Clips, jetPRIME transfection reagents were used as per manufacturer's protocol for 6 hrs. in 6 well plates. Briefly, for each well (total volume of 2 mL), 2 μg of DNA GQ Clips were mixed with jetPRIME transfection reagents in 1:2 ratio and incubated for 10 min. After transfection for 6 hrs., transfection media was replaced with new medium.

### Western Blotting

Cells were treated for 24 hours with media containing Clip sequence, scrambled sequence, or nanoparticle complexes. The cells were then washed three times with full growth media and total cellular protein was extracted from treated SH-SY5Y cells using a trizol reagent as per manufacturer's protocol. Protein lysate was separated by 15% SDS-PAGE. The proteins were detected by mouse monoclonal TH antibody (Sigma, T1299) at 1:200 dilution and GAPDH (G-9, sc-365062) antibody at 1:10000 dilution. Horseradish peroxidase-conjugated goat anti-mouse IgG (sc-2005) was used as secondary antibody at a dilution 1:1000. Proteins were visualized by Western Blotting Luminol Reagent (sc-2048) in a GE imaginer.

### Detection of Dopamine

Cells were transfected with DNA oligos as described above. After transfection, the cells were trypsinized, and the cellular components were precipitated with 0.1 M perchloric acid solution. The level of dopamine present in the supernatant was analyzed via HPLC equipped with an electrochemical detector. Briefly, the supernatant was syringe filtered through a 0.22 μm nylon membrane filter and placed into the autosampler vials for HPLC-ECD analysis on a Thermo Scientific Ultimate 3000 system consisting of an ESA Model 582 pump set at 0.5 mL/min solvent flow. C-18 reversed-phase column (Waters Corporation) was used for the analysis. Dopamine was detected with an electrochemical detector (Coulochem III, ESA) with a PEEK filter-protected 5011A analytical cell (ESA, 5 nA; guard electrode, 205 mV; analytical electrode, 250 mV). Chromatograms were recorded using Chromeleon® software, which also controlled the pump, autosampler, and detector. The HPLC solvent consisted of 15% v/v acetonitrile, 10% v/v methanol, 150 mM sodium phosphate buffer set to pH 5.3 with citric acid, 4.75 mM citric acid, and 50 μM EDTA. The HPLC solvents were vacuum degassed before use.

### Preparation of Gold Nanoparticles

Gold nanoparticles were synthesized following to a previously reported protocol of Beals, N. et al. The solution was then filter sterilized using a 0.2 μm cellulose acetate filter (Corning). Ten kDa membrane cut-off 50 ml centrifuge tubes were used to concentrate the nanoparticle solution to 2 ml. Particle size was then analyzed by UV-vis spectroscopy, TEM, and dynamic light scattering (DLS). A molar extinction coefficient of the 5 nm AuNP was used to obtain nanoparticle concentration via UV-vis spectroscopy (Cary 5000, Agilent).

### PEGylation of TrkB Aptamer

TrkB peptide aptamer (CENLYFQSGSMAHPYFAR) was purchased from Genscript (Piscataway, NJ, USA). The crude peptide was purified using reverse-phase C18 HPLC (put specifics, Flow rate, solutions, column size, solid phase, particle size). The purified peptide was conjugated to bismalemide (BM)-(PEG) in excess using the manufacturer's protocol (Pierce, Thermofisher). The BM(PEG)-peptide was purified using 1000 Da MWCO dialysis. Both the peptide and the BM(PEG) were examined for proper size using ESI-MS (FIGS. S3-6). A 1.5:1 molar ratio of BM(PEG)-peptide to 5 kDa SH-PEG-SH were mixed in water over 24 hrs. at room temperature to create the thioether product. The PEG-conjugated aptamer was purified via 3 kDa MWCO centrifuge tubes.

### Synthesis of the 5′GQ Clip Nanocomplexes

First, AuNP and PEG aptamers were mixed at a 1:1.5 molar ratio and shaken for 1 min. Afterwards, a 1:5:2 ratio by volume of AuNP-aptamer complex, 5 kDa SH-PEG-COOH 1 mM solution and 1 mM 5′GQ Clip DNA were mixed and shaken for one hour at 4° C. The nanocomplex was purified by dialysis with 100,000 kDa MWCO centrifuge tubes (Millipore). The resulting nanoparticle complex was added to increasing amounts of NaCl to test stability in salt. UV-Vis spectrophotometer was used to assess the AuNP peak at 515 nm for aggregation. (FIG. S6).

Detection of 5′GQ Clip release from Nanoparticle Complexes by Agarose Gel

The 5′-end-radiolabeled single-stranded oligonucleotides were prepared as described previously. The labelled Clip DNA was conjugated onto the nanoparticles with cold DNA to equal the normal nanoparticle loading. The nanoparticle samples were then incubated with 10 mM glutathione for various time points. The samples were analyzed on a 1% agarose gel. (FIG. S7). The gel was exposed to a phosphorimager screen and then visualized by Typhoon Phosphorimager FLA 9500 (GE Healthcare, Life Sciences).

### Cellular Uptake Detection by Confocal Fluorescence Microscopy

SH-SY5Y cells were seeded overnight at a density of 75,000 cells per well respectively in an 8-well chamber slide. Cells were then treated for 24 hours with media containing nanoparticles complexes conjugated with fluorescently labelled hexachlorofluorescein (HEX) 5′GQ Clip. The cells were then washed three times with full growth media and immediately analyzed for intracellular Dox distribution under an Olympus 1000× Confocal microscope.

### Animals

Adult male TH-GFP transgenic rats (245-300 g: Taconic Biosciences, USA) and Fischer 344 rats (240-300 g: Charles River, USA) were individually housed in Plexiglas cages (60×30×24 cm³). Rats were allowed approximately 7 days to acclimate to the colony after shipment before being handled for approximately 4 days before experiments were performed. Food and water were provided ad libitum. Studies were performed in accordance with the guidelines of the PHS Guide to the Care and Use of Laboratory Animals and approved by the Kent State University Institutional Animal Care and Use Committee.

### In Vivo Neuronal Uptake

5′GQ Clip nanoparticle was synthesized with fluorescently labelled hexachlorofluorescein (HEX) 5′GQ Clip. Fischer 344 rats were briefly anesthetized under Isoflurane and intravenously injected via the tail vein with 10 μg of HEX-labeled 5′GQ Clip nanoparticle. Twenty-four hours later, animals were deeply anesthetized with pentobarbitol and transcardially perfused with 250 ml saline followed by 400 ml 4% paraformaldehyde. Rat brains were harvested, post-fixed for 24 h in paraformaldehyde prior to placement in 30% sucrose and sliced into 20 μm sections spanning the region of the substantia nigra containing the targeted dopaminergic neurons. Confocal microscopy was used where neurons were stained with NeuN to observe HEX positive neurons.

### Modulation of TH-GFP in a Transgenic Mouse Model

Two groups of TH-GFP rats (n=3) were treated intravenously via the tail vein with 10 μg (in reference to loaded DNA) of 5′GQ Clip nanoparticle or scrambled nanoparticle and perfused 24 hours later as described previously. Rat brains were harvested, fixed and sliced into 20 μm sections spanning the region of the substantia nigra containing the targeted dopaminergic neurons. Confocal microscopy was used to observed GFP expression. Image J software was used to quantify the fluorescence between the two treatments. The total area of fluorescence was normalized against the number of nuclei (>1500 nuclei per image).

RNA Isolation and qPCR

RNA was isolated and purified from the brain regions collected using trizol reagent as per manufacturer's protocol. Purified RNA was checked for quality and quantity on a Nanodrop. Only those samples that had 260/280 and 260/230 ratios greater than 1.7 were processed further. RNA was stored at −80° C. until it was assayed. The cDNA was synthesized using qScript™ cDNA SuperMix (Quanta Biosciences). The glyceraldehyde 3-phosphate dehydrogenase (GAPDH) and GFP mRNAs were subjected to RT-qPCR using a Perfecta® SYBR® Green Super Mix (Quanta Biosciences) on an Eppendorf Mastercycler® RealPlex2 in the presence of the appropriate set of primers. The relative mRNA levels were estimated by the comparative Ct method (Livak method).

### Statistical Analyses

If not stated otherwise, results are mean values±standard error of mean (SEM) of at least three independent experiments, or results show one representative experiment of a minimum of three. Statistical analyses were performed on all available data. Unless otherwise mentioned, statistical significance was determined using the two-tailed Student's t test with p values s 0.05 considered statistically significant.

### Example 1—GQ Clip Sequences Bind and Influence G-Quadruplex Formation within the Human Tyrosine Hydroxylase Promoter

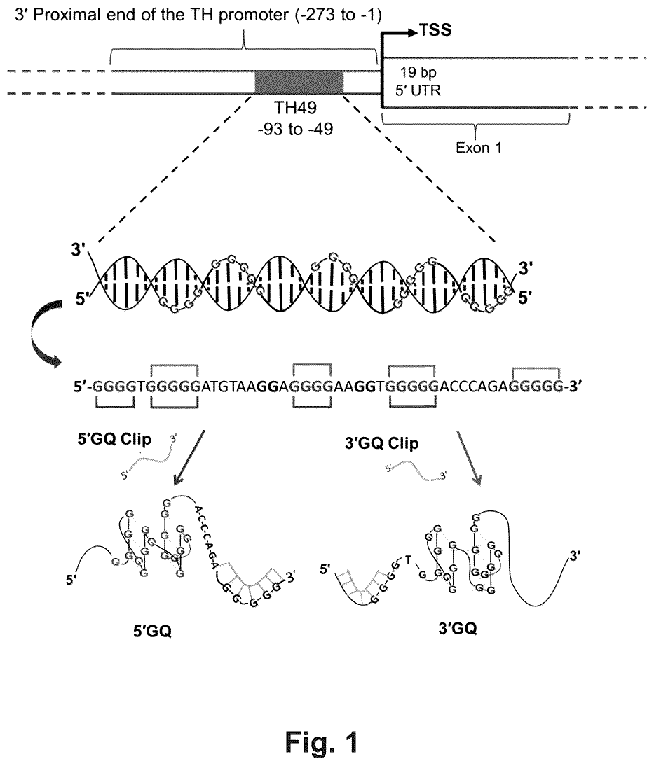

The TH49 sequence within the human TH promoter contains seven G-stretches (I-VII) and adopts two major GQ structures named 5′GQ and 3′GQ, which play distinct roles in controlling TH transcription. In the two different sets of G-quadruplex (GQ) structures (5′GQ and 3′GQ), the 5′GQ uses G-stretches I, II, IV and VI in TH49 which enhances TH transcription, while the 3′GQ utilizes G-stretches II, IV, VI and VII which represses transcription. However, G-stretches III and V are not involved for the formation of either 5′GQ or 3′GQ (FIG. 1). While the 5′GQ structure enhances the cellular TH transcription, 3′GQ structure acts as a repressive element. Here, two different DNA molecules were designed, named “GQ Clips”, to specifically block the formation of either the 5′GQs or the 3′GQs within TH49 to favor the formation of one or the other GQ. For example, the 5′GQ Clip can bind with 3′ end of the sequence where G-stretch VII is located. Given that G-stretch VII is essential for the formation of 3′GQ, binding of 5′GQ Clip blocks the 3′GQ structure formation and in turn favors the formation of the 5′GQ structure. Meanwhile, the 3′GQ Clip can bind with 5′ end of the sequence where G-stretch I is located. Thus, the 3′GQ Clip blocks the 5′GQ structure formation in the TH promoter since 5′GQ structure cannot be formed without G-stretch I (FIG. 1). The 3′ and 5′ GQ Clips are 20 and 21 nucleotides-long (oligos) respectively, with phosphorothioate modifications at each end to increase stability against nucleases.

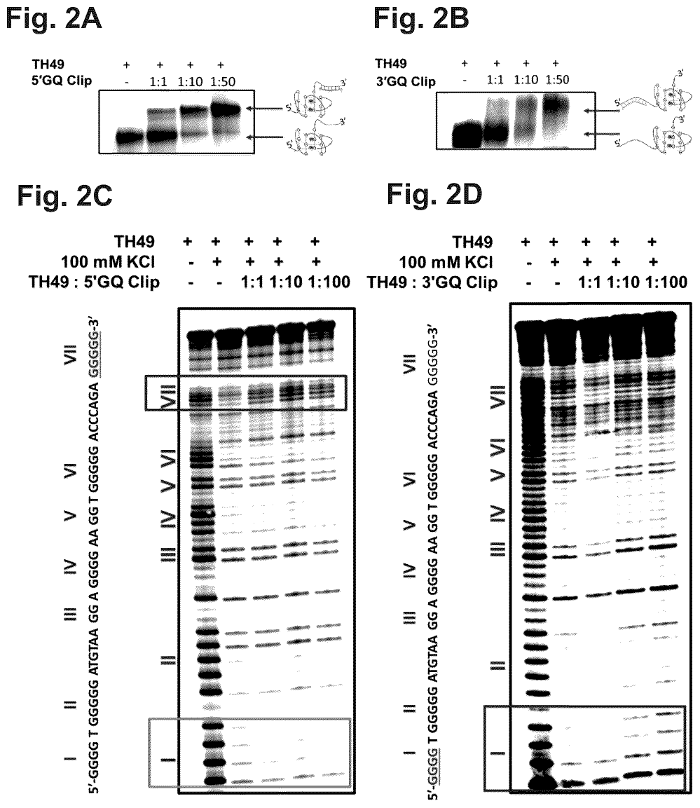

A non-denaturing electrophoretic gel mobility shift assay (EMSA) was performed to determine whether the GQ Clips can bind to the TH49 sequence and interfere with the GQ folding, which should result in changes in the migration patterns (FIGS. 2A and 2B). The ability of both GQ Clips (5′GQ Clip and 3′GQ Clip) to interact with TH49 was tested with increasing amounts of GQ Clips in the presence of 100 mM K⁺. As shown in FIG. 2A, the leftmost lane shows the mobility of the native GQ adopted by the TH49 sequence in the absence of GQ Clips. As expected, in the presence of increasing amount of 5′GQ Clip, another DNA species emerges, which has a slower mobility than the native TH49 GQs representing complexation of the 5′GQ Clip with TH49 GQ. The binding is more prominent as the concentration of the 5′GQ Clip increases, moving the equilibrium towards the slower moving band (FIG. 2A, Lanes 2-4). Binding of the 3′GQ Clip to wtTH49 was also observed in FIG. 2B, displaying a similar affinity but for the 5′ end of the TH49 sequence.

Once initial binding of the GQ Clips with the TH49 sequence was confirmed, dimethyl sulfate (DMS) structural mapping was performed to determine the capability of the GQ Clips to block the targeted GQ within the TH49 and in turn, promote the formation of the other GQ. The N7 of guanine in a B-form duplex DNA is not involved in Watson-Crick hydrogen bonding in GC base pairs. Therefore, the N7 position of guanine in the duplex and single-stranded DNA can be methylated by DMS, leading to subsequent DNA strand cleavage upon treatment with piperidine. However, in the context of a GQ, N7 of each guanine is hydrogen-bonded to N2 of the neighboring guanine to form a tetrad. Consequently, the N7 of such guanines are protected from the DMS methylation and the subsequent piperidine cleavage. The DMS footprinting of TH49 DNA in the presence of increasing 5′GQ Clip concentration is shown in FIG. 2C. The leftmost lane, a no KCl control, displayed an unstructured sequence where all guanines in the sequence appeared to be susceptible to DMS modification. This was expected since the formation of GQ requires K⁺ as a monovalent cation for stabilization. This is the opposite of lane 2 as the structured TH49 was observed due to the presence of 100 mM K⁺. All five G-stretches (I, II, IV, VI, and VII) that are involved in the formation of the GQ structures are highly protected. However, the gradual increase of the intensity of bands corresponding to G-stretch VII was witnessed in the presence of 5′GQ Clip (FIG. 2C, lanes 3-5), implying increasing deprotection of this particular G-stretch. Given that the G-stretch VII is only involved in the formation of 3′GQ structure, this observation strongly suggested that the binding of 5′GQ Clip to TH49 blocked the 3′GQ from adopting its conformation and as a result, the guanines in the G-stretch VII are increasingly modified by DMS. On the other hand, the intensity of bands corresponding to G-stretch I gradually increased with increasing 3′GQ Clip concentration in FIG. 2D. Since G-stretch I is only involved in the formation of 5′GQ structure, this observation provided clear evidence for the induction of 3′GQ structure by the 3′GQ Clip. Overall, the DMS assay provided strong evidence that the 5′GQ Clip favoured the formation of the 5′GQ structure by blocking the adoption of 3′GQ in the G-rich region of the TH promoter and, on the other hand, the 3′GQ Clip bound with the 5′ end of the TH49 sequence and blocked the 5′GQ formation.

To further assess the ability of GQ Clips to modulate target GQ formation, mutated TH49 sequences (see Table 1, above) were designed. The mutated TH49 sequences are such that, if the targeted GQ modulation is working as proposed, TH49 5′GQ mutant should not be able to form a GQ when treated with 5′GQ Clip, and TH49 3′GQ mutant should not be able to form a GQ upon the treatment with 3′GQ Clip (FIG. S3). Indeed, that was exactly what was observed. (Results not shown). When TH49 5′GQ mutant was treated with 5′GQ Clip sequence, it lost its GQ feature (CD peak shifted from 263 nm to 270 nm), but there was no shift with the 3′GQ Clip treatment. On the other hand, the treatment of TH49 3′GQ mutant with 3′GQ Clip caused the reduction in the peak intensity of the GQ signal but that was not the case with the treatment of TH49 3′GQ mutant with 5′GQ Clip. To further test whether the reduction in the GQ peak intensity (263 nm) of TH49 3′GQ mutant upon treatment with 3′GQ Clip was truly due to their binding, their CD behavior in two different Clip concentrations were analyzed. A concentration dependent reduction was observed in peak intensity of TH49 3′GQ mutant indicating the ability of 3′GQ Clip to bind to the target region of TH49 and modulate its GQ formation.

The data presented herein, namely, the electromobility gel shift assay, the DMS footprints, and CD data demonstrated the ability of engineered Clip sequences to modulate target GQ formation in TH49 sequence.

### Example 2—Cellular Tyrosine Hydroxylase Expression Controlled with the Treatment of GQ Clip Sequences

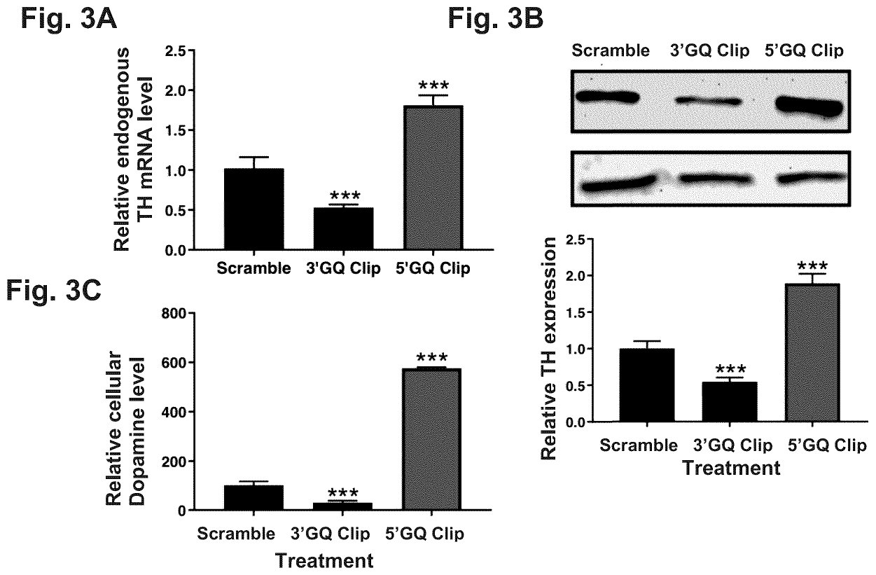

Tests were undertaken to determine whether the in vitro modulation of TH49 GQs by DNA Clips can be replicated in the cells. Targeting the GQ region located proximal to the transcription initiation site (TIS) within the TH promoter should modulate the endogenous TH mRNA level. To investigate this, the GQ Clips (Table 1) in the human neuroblastoma (SH-SH5Y) cells were transfected. As shown in FIG. 3A, the 3′GQ Clip reduced the endogenous TH mRNA level by 2-fold as compared to cells transfected with a scrambled control. It is believed that this was accomplished by blocking the 5′GQ structure, which in turn favors the formation of 3′GQ. Importantly, an increase of endogenous TH mRNA level (2-fold) was detected in cells that were treated with 5′GQ Clip (FIG. 3A). Blocking of 3′GQ structure favors the formation of the 5′GQ structure which enhances TH transcription resulting in the synthesis of more mRNA.

Next, Western blot was used to measure TH protein expression in SH-SY5Y cells that were treated with the GQ Clips (FIG. 3B). A 30% reduction in the TH protein expression was detected in the cells treated with the 3′GQ Clip and the 5′GQ Clip increased TH expression by 60% as compared to the scrambled control. Thus, the RT-qPCR and Western blot results confirmed the modulation of TH transcription by GQ Clips presumably by ensuring the formation of specific GQ structures in the TH promoter which affects the TH protein production in the SH-SY5Y cells. In addition to the scramble control, a second control was used, which was termed as promoter control sequence and was designed to bind within the TH promoter upstream of the targeted TH GQ region while not hindering other important stretches that are known to involve in the regulation of transcription. (Table 1, above).

### Example 3—Dopamine Synthesis in the Neuronal Cells Modulated with GQ Clips

Since the TH enzyme catalyzes the rate-limiting step in the catecholamine biosynthesis pathway, it was important to investigate whether the modulation of TH expression by the GQ Clips affects the synthesis of dopamine, a neurotransmitter which is produced in the subsequent step. For this, cellular analytes were isolated from the Clip-treated cells, and the relative presence of dopamine was measured using HPLC separation followed by electrochemical detection. Significant changes in the cellular dopamine level were observed in the GQ Clip treated cells (FIGS. 3C and S1). Treatment with the 3′GQ Clip displayed a 3-fold reduction in the dopamine level, while the 5′GQ Clip treated cells showed a 5-fold increase. This suggested a novel therapeutic DNA sequence, which can alter not only cellular TH mRNA and protein but also cellular dopamine levels by controlling the rate-limiting enzyme in the catecholamine biogenesis pathway using rationally designed exogenous agents. Several neurological disorders are associated with abnormal dopamine levels, making this a viable approach for correcting the dopamine level to elicit a therapeutic response.

### Example 4—Synthesis and Characterization of a Nanoparticle Delivery System to Improve the Therapeutic Potential of 5′GQ Clip

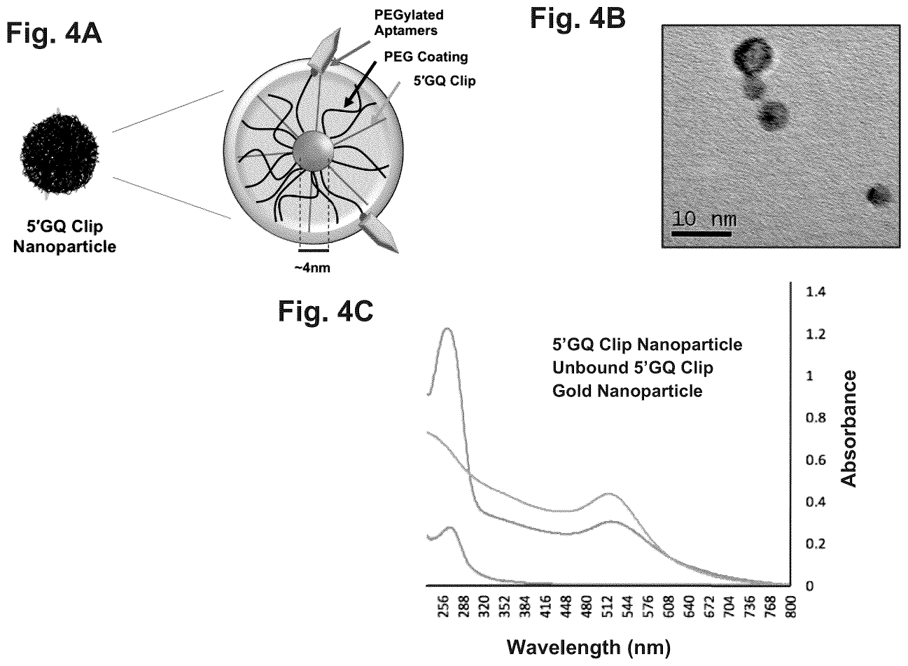

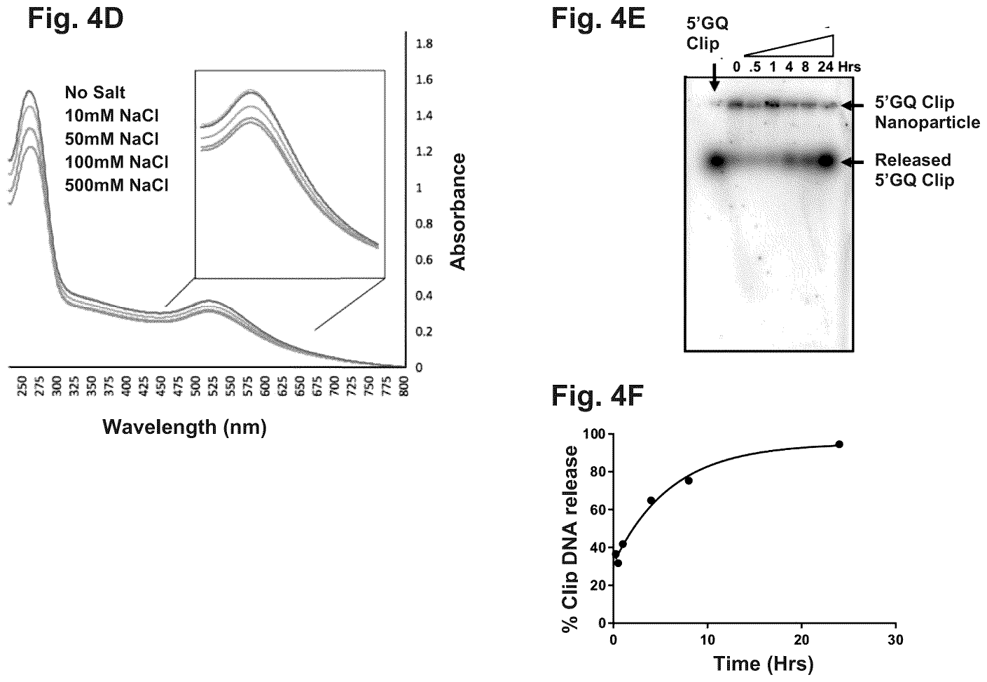

Based on the in vitro and in cellulo success of modulating TH expression using the GQ Clips, an approach was advanced to develop an in vivo model for the potential treatment of neurological diseases, such as Parkinson's disease where the 5′GQ Clip could be used to increase dopamine synthesis. Transfecting DNA molecules for therapeutic purposes is not a viable option for delivering nucleic acid agents into the neurons of an animal brain. Thus, to take full therapeutic advantage of the GQ Clips, a nanoparticle complex was designed to create a translation method for targeted neuronal uptake. To accomplish this, a nanoparticle complex (5′GQ Clip nanoparticle) was synthesized, comprising of polyethylene glycol (PEG)-coated gold nanoparticle (AuNP) as a central tethering agent, PEGylated tyrosine receptor kinase B (TrkB), and transferrin receptor 1 (Tfr1) aptamers, and the 5′GQ Clip molecules (FIG. 4A, S7-S13, Tables S1 and S2). Targeting aptamers have been paired with nanotechnology to particularly bypass negative pharmacokinetic characteristics previously associated with nucleic acid delivery. Utilizing this strategy, it is believed the synthesized dual aptamer nanoparticle conjugate can be used for systemic injection of the 5′GQ Clip nanoparticle which will cross the blood-brain barrier and be selectively taken up by dopaminergic neurons. For this purpose, previously reported nucleic acid aptamer for binding to Tfr1 for brain uptake was used. To target dopaminergic neurons, a previously reported 18 amino acid sequence targeted to the TrkB receptor that is highly expressed on the cell surface was used. Transmission electron microscopy (TEM) images of 5′GQ Clip nanoparticle displayed the AuNPs were between 4.5+/−1 nm in size, which are advantageous for the passive excretion from the brain and the body based on selective sizing (FIG. 4B). UV spectroscopy and gel electrophoresis were used to quantify DNA loading and stability of the 5′GQ Clip nanoparticle, monitored at 260 nm and 515 nm respectively (FIGS. 4C and 4D). The characteristic AuNP 515 nm peak was determined to remain the same with increasing concentrations of NaCl indicating the enhanced stability of the nanoparticle complex, whereas a redshift would have indicated nanoparticle aggregation.

By using thiolated nucleic acids for AuNP conjugation, an inherent intracellular release mechanism was created where elevated intracellular levels of glutathione (GSH) can compete and replace ligands at the AuNP surface. It was found that the 5′GQ Clip released in a time-dependent manner in the presence of 10 mM GSH, which is the average intracellular concentration (FIGS. 4E and 4F). The bands of the gel in FIG. 4E were quantitated and plotted to generate a release curve (FIG. 4F). The use of radiolabeled DNA allowed the detection of two distinct 5′GQ Clip bands. The upper slower-migrating band correlated with the DNA still attached to the nanoparticle, while the faster-migrating band corresponded to the released 5′GQ Clip. Within the first hour, 50% of the 5′GQ Clip was released from the nanoparticle complex and after 24 hours nearly all of the DNA was released.

### Example 5—Cellular Uptake of 5′GQ Clip Nanoparticle Dependent on TrkB Aptamer

To visualize the improved uptake of the 5′GQ Clip sequence while attached to the nanoparticle complex, a fluorescently labelled (FAM) 5′GQ Clip sequence was used. Confocal microscopy images illustrate the uptake of the 5′GQ Clip sequence (no transfection), a no aptamer 5′GQ Clip nanoparticle complex, and the 5′GQ Clip nanoparticle complex containing the TrkB aptamer in SH-SY5Y cells (FIG. 5A). The 5′GQ Clip nanoparticle complex was observed to have dramatically higher cellular uptake when compared to the other two treatments. Specific binding to the receptor via the TrkB aptamer is expected to initiate receptor-mediated endocytosis which helps the internalization of these larger complexes. These images highlighted not only the ability of the 5′GQ Clip nanoparticle to enter the cells but also the incredibly limited uptake exhibited by both the free DNA and the no aptamer nanoparticle. Both DNA and the no aptamer 5′GQ Clip nanoparticle complex are large and highly charged, presumably making it challenging to enter the cell through non-receptor mediated endocytosis mechanisms.

### Example 6—Clip Nanoparticle Improved the Functional Efficiency of 5′GQ Clip in Human Neuronal Cells

A large obstacle in nucleic acid therapeutics and drug delivery is cellular specificity and uptake. Oligonucleotide drugs, such as small interfering RNA (siRNA), anti-sense DNA, and micro-RNA are handicapped by poor cellular uptake due to high overall net negative charge and large size making pinocytosis less effective. To determine the possible therapeutic advantage of the 5′GQ Clip nanoparticle, initially, the change in endogenous TH mRNA and protein levels were investigated. As seen before, the 5′GQ Clip increased TH expression by 2-fold, but the 5′GQ Clip nanoparticle caused a 9-fold increase when both were treated at the same concentration of 270 nM (FIG. 5B). Western blot analysis of the cell lysates from 5′GQ Clip only and 5′GQ Clip nanoparticle-treated cells revealed similar results where a 2-fold increase in TH protein expression occurred with the treatment of the 5′GQ Clip only, while the 5′GQ Clip nanoparticle contributed to an average of a 6.5-fold increase in TH protein expression (FIG. 5C). Two other nanoparticle complexes were also synthesized as controls to test the necessity for the simultaneous presence of the 5′GQ Clip and TrkB aptamer on the 5′GQ Clip nanoparticle for it to be fully functional. The first nanoparticle complex replaced the 5′GQ Clip with a scrambled DNA sequence (scrambled Clip nanoparticle) but still possessed the TrkB aptamer. The second complex was a no aptamer 5′GQ Clip nanoparticle that had the 5′GQ Clip therapeutic, PEG, and AuNP but no TrkB aptamer. Western blot analysis of the cell lysate after the treatment displayed the scrambled Clip nanoparticle had no relative difference in protein expression compared to the untreated control, while the no aptamer complex had a slight increase (1.5-fold) (FIG. S9).

### Example 7-5′GQ Clip Nanoparticle Increased TH Expression in a Toxin-Induced PD Cell Model

Parkinson's disease (PD) is one of the most common and life-altering neurodegenerative diseases, involving the dysregulation of dopamine due to neuronal death. 6-hydroxydopamine (60HDA) is a neurotoxin that when supplemented in cellular growth media, promotes intracellular ROS and cell death mimicking PD. To examine the effect of how the 5′GQ Clip nanoparticle can modulate TH in a neurotoxin mediated stressed environment, SH-SY5Y cells were treated with 5 μM 60HDA with and without the 5′GQ Clip nanoparticle. In the presence of the 5′GQ Clip nanoparticle, the TH mRNA level was 25-fold higher in relation to the neurotoxin only treated cells (FIG. 5D). Similarly, TH protein expression of the 5′GQ Clip nanoparticle treated cells with 60HDA was 3-fold higher than the neurotoxin treated control (FIG. 5E). Analyses of qRT-PCR and Western blot were further performed with increasing 5′GQ Clip nanoparticle concentrations (270 nM and 540 nM) to observe if there was a dose-dependent response to the treatment. A 2-fold increase in 5′GQ Clip nanoparticle concentration indeed enhanced TH mRNA expression by ˜2-fold was observed. In relation to the no treatment control and 60HDA treatment, the 540 nM concentration saw a 5-fold and 20-fold increase in TH mRNA respectively (FIG. S10). In terms of protein expression, a ˜2-fold increase was observed at 540 nM compared to the 270 nM treatment (FIG. S11). This highlights that the system offers potential controllability in increasing TH expression as needed. Subsequently, SH-SY5Y cells were incubated with increasing concentrations of 60HDA with and without 540 nM 5′GQ Clip nanoparticle to ensure that the nanoparticle did not perpetuate the cytotoxic effect of the disease state. The cellular viability assay displayed the opposite effect where the nanoparticle complex decreased 60HDA toxicity by 1.5-fold (25 μM to 40 μM) (FIG. S12). Thus, it was clear that the 5′GQ Clip nanoparticle provided a level of protection against the 60HDA-mediated toxicity.

### Example 8—TH-GFP Modulated by 5′GQ Clip Nanoparticle In Vivo

To achieve the desired effect on neurological disorders, targeted neuronal uptake is necessary for therapeutic efficacy, specifically being able to localize into neurons of the substantia nigra. To examine this, the 5′GQ Clip nanoparticle was subjected to in vivo experimentation to test if the design of the drug delivery complex could indeed reach the dopaminergic regions of an animal brain. Rats were intravenously injected via the tail vein with 10 μg of hexachlorofluorescein (HEX) labeled 5′GQ Clip nanoparticle and sacrificed after 24 Hrs. Rat brains were harvested, fixed and sliced into 20 μm sections that contained the substantia nigra region containing the targeted dopaminergic neurons.

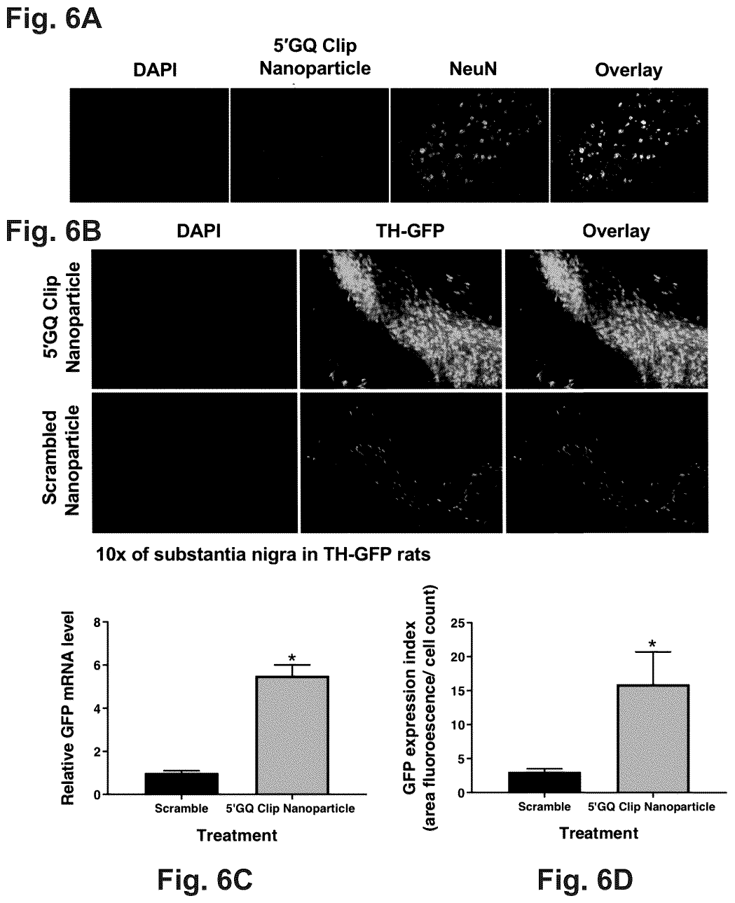

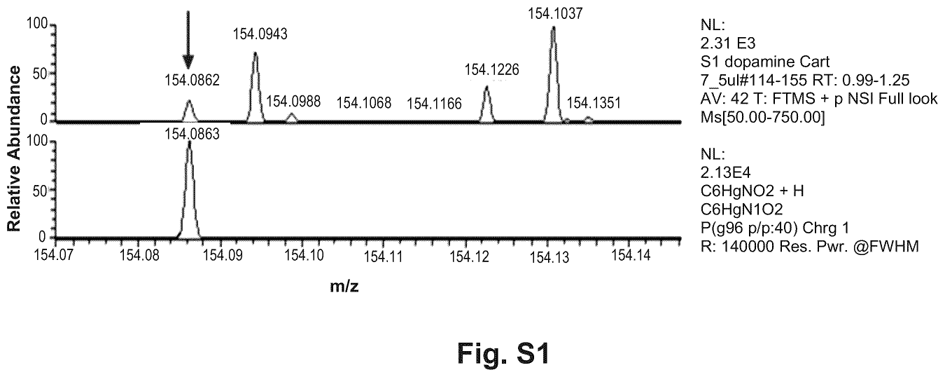

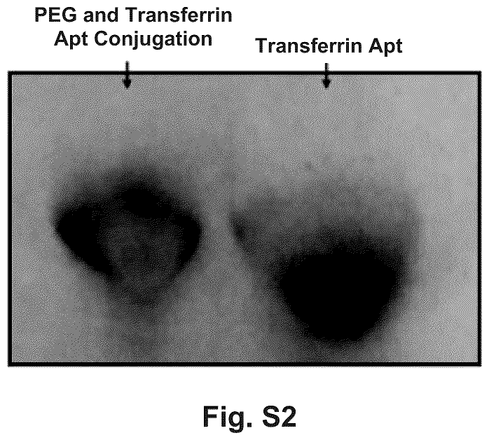

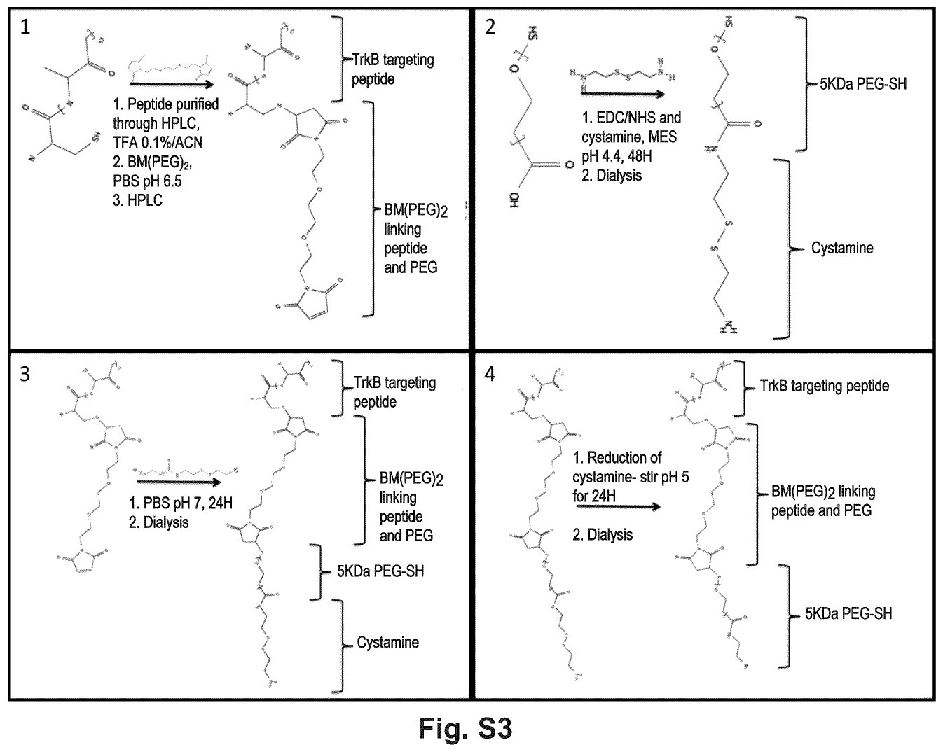

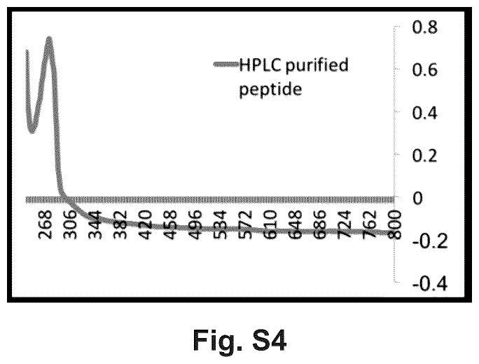

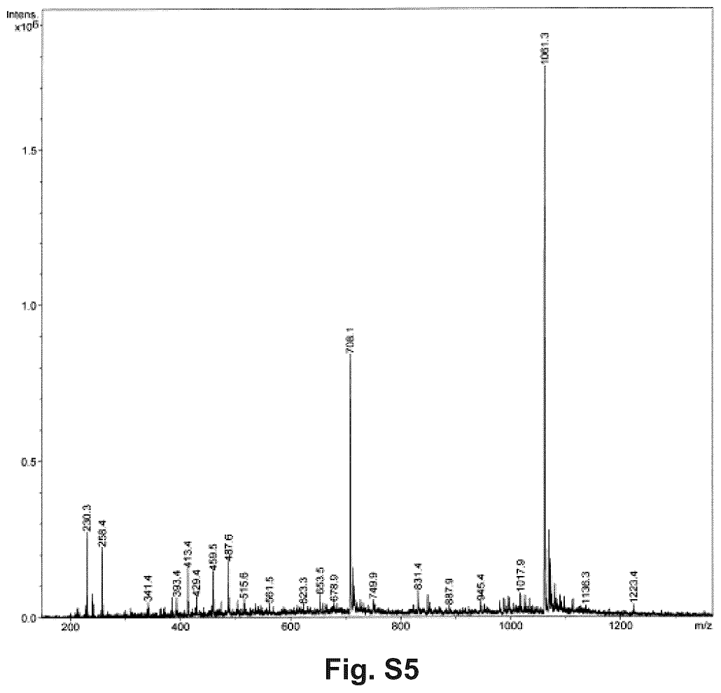

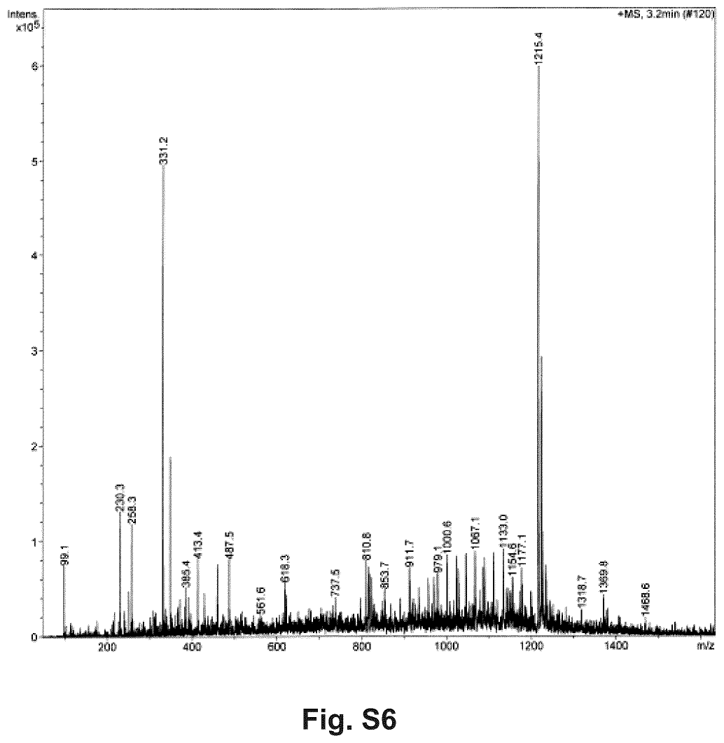

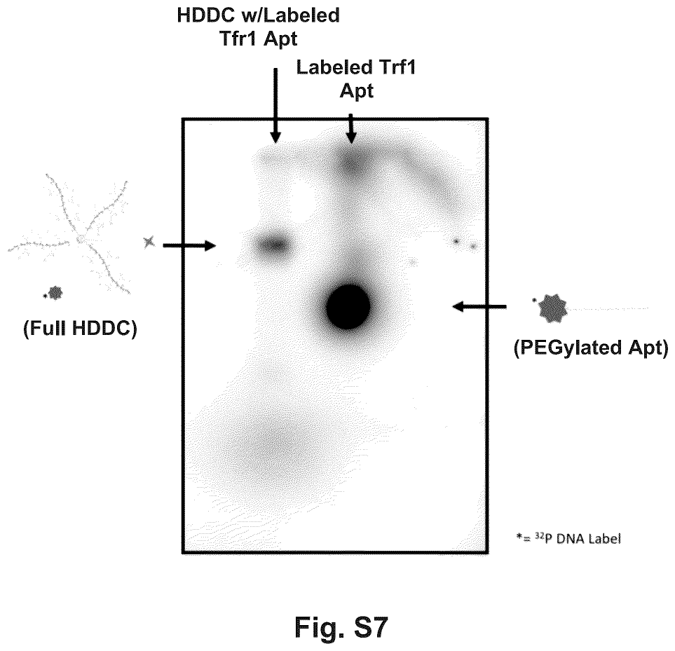

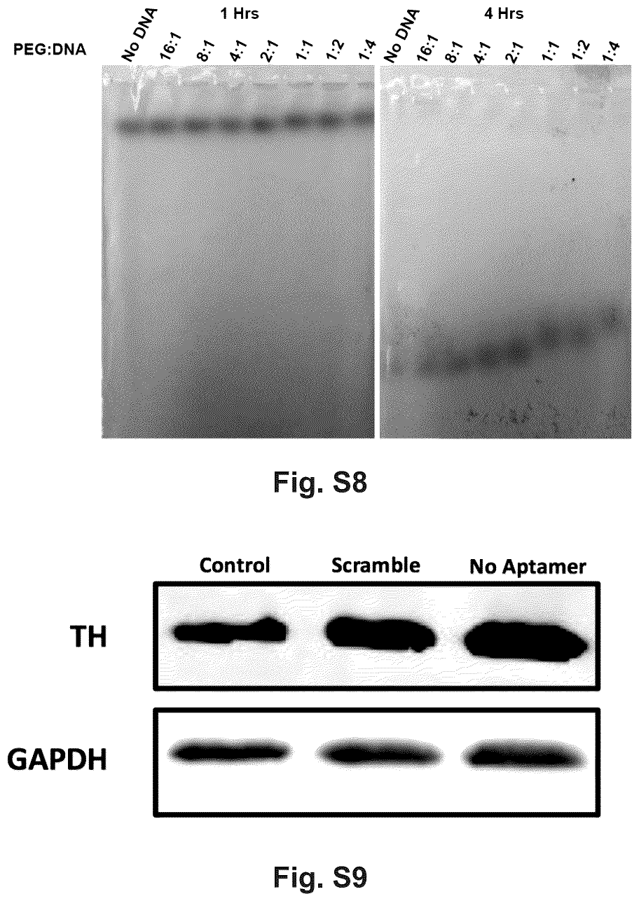

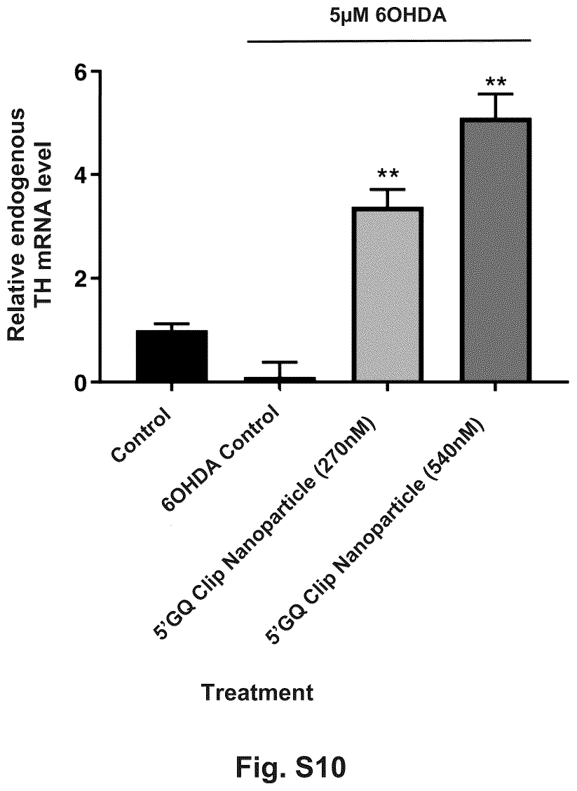

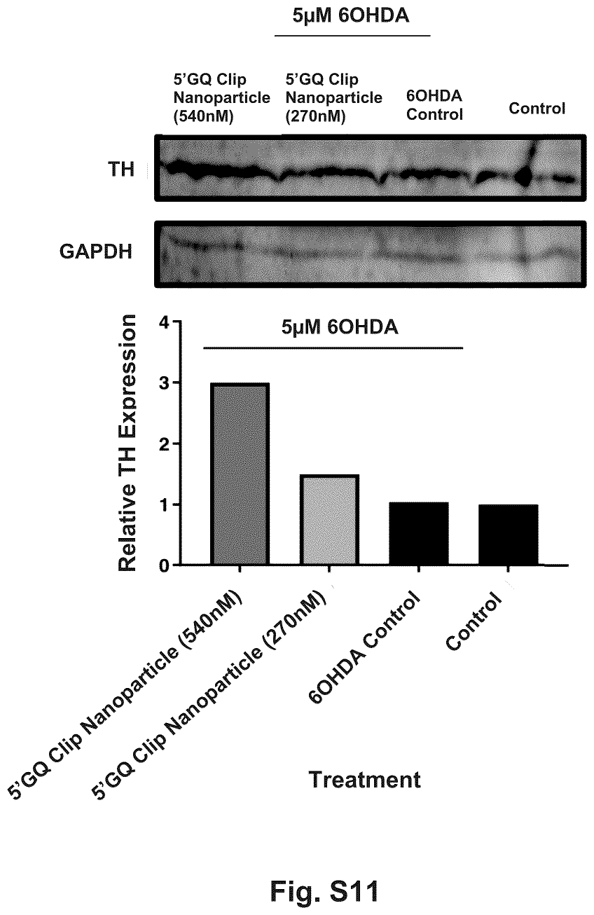

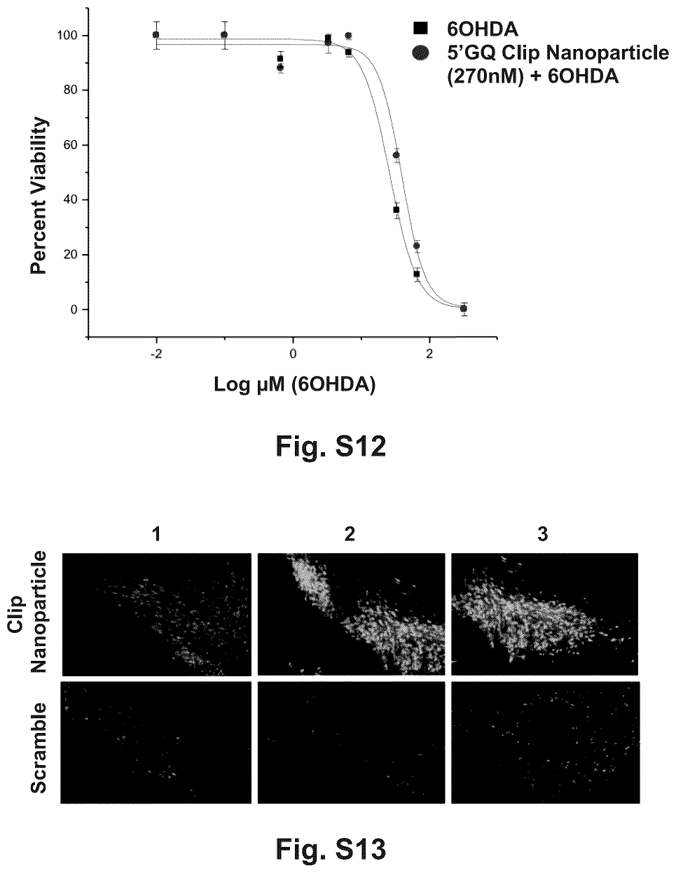

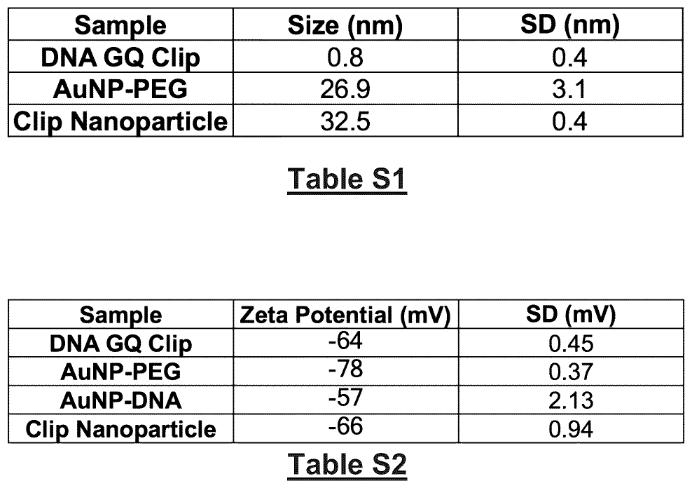

Using Confocal microscopy, HEX staining was observed across the slices and higher magnification images showed HEX staining colocalized with neurons (NeuN-positive cells) (FIG. 6A). This demonstrated the ability of the 5′GQ Clip nanoparticle to not only enter the brain but also target the dopaminergic neurons (as seen in FIG. 5A). This was a highly encouraging result given that many neurological drugs encounter the common lacunae of the inability to cross the blood brain barrier.

With successful uptake into neurons, the therapeutic relevance of the 5′GQ Clip nanoparticle was next assessed in a transgenic TH-GFP rat model. The human TH promoter sequence is not conserved in the rat or mouse genome, so we used a commercially available model developed by the Lacovitti lab. This model utilized GFP expression driven by the human TH gene promotor that encompasses the 49 nucleotide (nt) region targeted by the 5′GQ Clip. It has been previously reported that GFP expression is exhibited with high specificity to dopaminergic regions including the substantia nigra and striatum. This model allowed for modulation of reporter GFP expression using our 5′GQ Clip nanoparticle. The TH-GFP rats were treated intravenously with 10 μg of 5′GQ Clip nanoparticle with 5′GQ Clip or scrambled DNA and sacrificed after 24 Hrs. Specific brain areas were studied using immunofluorescence staining and imaging of the respective tissue sections. The 5′GQ Clip nanoparticle caused a dramatic increase in GFP expression, suggesting the 5′GQ Clip's ability to target the human TH promoter thereby increasing the expression of the linked transgene (FIGS. 6B and 6D and S13). Neurons within the entirety of substantia nigra displayed higher fluorescence, aligning with the data in FIG. 6A where 5′GQ Clip nanoparticle uptake was observed in the majority of the neurons. ImageJ software was used to further assess the changes in fluorescence between the two treatments. The total area of fluorescence was normalized against the number of nuclei (>1500 nuclei per image) and displayed a robust 5-fold increase in GFP fluorescence.

Further analysis was performed to examine the mRNA levels in the presence of each treatment from brain slices containing the substantia nigra. qRT-PCR showed the 5′GQ Clip nanoparticle-treated rats had over a 5-fold increase in mRNA GFP expression when compared with scrambled Clip nanoparticle treated rat tissue (FIG. 6C). Successful increase in GFP mRNA and protein expression in vivo establishes that the 5′GQ Clip nanoparticle complex can not only reach neurons within the substantia nigra, but also can be potentially used as a viable system to modulate TH and therefore dopamine in an in vivo system for the treatment of Parkinson's disease and other dopaminergic diseases.

The examples above demonstrated that the human tyrosine hydroxylase promoter harbors a unique biochemical region that allows precise control of endogenous TH expression and therefore dopamine production providing an opportunity to improve upon current therapeutic approaches. This precise control is inherent to the ability of the GQ Clips to bind and regulate the specific GQ formation within the TH promoter. Nucleic acid therapeutics provide a high level of specificity, but major challenges exist in the realm of cellular targeting and uptake. Here, a novel DNA-based approach was introduced, with a translational nanoparticle delivery system, to control specific GQ formation within the human TH promoter and in turn, the TH expression culminating in up or down regulation of dopamine production. These in vitro and in vivo studies described herein provide the groundwork for the advancement of GQ Clip centric neurological disease therapy, such as Parkinson's Disease, where the 5′GQ Clip could be used to enhance dopamine level.

While in accordance with the patent statutes, the best mode and preferred embodiment have been set forth, the scope of the invention is not limited thereto, but rather by the scope of the attached claims.

General discussions herein describe background work in the field and are based on the following references.

### BACKGROUND REFERENCES

1. Ullrich, R.; Hofrichter, M., Enzymatic hydroxylation of aromatic compounds. Cell. Mol. Life Sci. 2007, 64 (3), 271-293.
2. Molinoff, P. B.; Axelrod, J., Biochemistry of catecholamines. Annu. Rev. Biochem. 1971, 40, 465-500.
3. Tripp, G.; Wickens, J. R., Neurobiology of ADHD. Neuropharmacology 2009, 57, 579-589.
4. Arnsten, A. F., Catecholamine regulation of the prefrontal cortex. J. Psychopharmacol. 1997, 11 (2), 151-162.
5. Assadi, S. M.; Yucel, M.; Pantelis, C., Dopamine modulates neural networks involved in effort-based decision-making. Neurosci. Biobehav. Rev. 2009, 33 (3), 383-393.
6. Anisman, H.; Zacharko, R. M., Behavioral and neurochemical consequences associated with stressors. Ann. NY Acad. Sci. 1986, 467, 205-225.
7. Brooks, D. J.; Piccini, P., Imaging in Parkinson's disease: the role of monoamines in behavior. Biol. Psychiatry 2006, 59 (10), 908-918.
8. Daubner, S. C.; Le, T.; Wang, S., Tyrosine hydroxylase and regulation of dopamine synthesis. Arch. Biochem. Biophys. 2011, 508 (1), 1-12.
9. Banerjee, K.; Wang, M.; Cai, E.; Fujiwara, N.; Baker, H.; Cave, J. W., Regulation of tyrosine hydroxylase transcription by hnRNP K and DNA secondary structure. Nat. Commun. 2014, 5 (5769).
10. Hwang, O., Role of oxidative stress in Parkinson's disease. Exp. Neurobiol. 2013, 22 (1), 11-17.
11. Parkkinen, L.; O'Sullivan, S. S.; Collins, C.; Petrie, A.; Holton, J. L.; Revesz, T.; Lees, A. J., Disentangling the relationship between lewy bodies and nigral neuronal loss in Parkinson's disease. J. Parkinsons Dis. 2011, 1 (3), 277-286.
12. Tabrez, S.; Jabir, N. R.; Shakil, S.; Greig, N. H.; Alam, Q.; Abuzenadah, A. M.; Damanhouri, G. A.; Kamal, M. A., A synopsis on the role of tyrosine hydroxylase in Parkinson's disease. CNS Neurol. Disord. DR 2012, 11 (4). 395-409.
13. Farhath, M. M.; Thompson, M.; Ray, S.; Sewell, A.; Balci, H.; Basu, S., G-Quadruplex-Enabling Sequence within the Human Tyrosine Hydroxylase Promoter Differentially Regulates Transcription. Biochemistry 2015, 54 (36), 5533-5545.
14. Williamson, J. R.; Raghuraman, M. K.; Cech, T. R., Monovalent cation-induced structure of telomeric DNA: the G-quartet model. Cell 1989, 59 (5), 871-880.
15. Yang, D.; Okamoto, K., Structural insights into G-quadruplexes: towards new anticancer drugs. Future Med. Chem. 2010, 2 (4), 619-646.
16. Qin, Y.; Hurley, L. H., Structures, folding patterns, and functions of intramolecular DNA G-quadruplexes found in eukaryotic promoter regions. Biochimie 2008, 90 (8), 1149-1171.
17. Qin, Y.; Rezler, E. M.; Gokhale, V.; Sun, D.; Hurley, L. H., Characterization of the G-quadruplexes in the duplex nuclease hypersensitive element of the PDGF-A promoter and modulation of PDGF-A promoter activity by TMPyP4. Nucleic Acids Res. 2007, 35 (22), 7698-7713.
18. Sun, D.; Guo, K.; Rusche, J. J.; Hurley, L. H., Facilitation of a structural transition in the polypurine/polypyrimidine tract within the proximal promoter region of the human VEGF gene by the presence of potassium and G-quadruplex-interactive agents. Nucleic Acids Res. 2005, 33 (18), 6070-6080.
19. Guo, K.; Gokhale, V.; Hurley, L. H.; Sun, D., Intramolecularly folded G-quadruplex and i-motif structures in the proximal promoter of the vascular endothelial growth factor gene. Nucleic Acids Res. 2008, 36 (14), 4598-4608.
20. Siddiqui-Jain, A.; Grand, C. L.; Bearss, D. J.; Hurley, L. H., Direct evidence for a G-quadruplex in a promoter region and its targeting with a small molecule to repress c-MYC transcription. Proc. Natl.l Acad. Sci. USA 2002, 99 (18), 11593-11598.
21. Cogoi, S.; Paramasivam, M.; Spolaore, B.; Xodo, L. E., Structural polymorphism within a regulatory element of the human KRAS promoter: formation of G4-DNA recognized by nuclear proteins. Nucleic Acids Res. 2008, 36 (11), 3765-3780.
22. Dexheimer, T. S.; Sun, D.; Hurley, L. H., Deconvoluting the structural and drug-recognition complexity of the G-quadruplex-forming region upstream of the bcl-2 P1 promoter. J. Am. Chem. Soc. 2006, 128 (16). 5404-5415.
23. Dai, J.; Chen, D.; Jones, R. A.; Hurley, L. H.; Yang, D., NMR solution structure of the major G-quadruplex structure formed in the human BCL2 promoter region. Nucleic Acids Res. 2006, 34 (18), 5133-5144.
24. Patel, D. J.; Phan, A. T.; Kuryavyi, V., Human telomere, oncogenic promoter and 5′-UTR G-quadruplexes: diverse higher order DNA and RNA targets for cancer therapeutics. Nucleic Acids Res. 2007, 35 (22), 7429-7455.
25. Balasubramanian, S.; Hurley, L. H.; Neidle, S., Targeting G-quadruplexes in gene promoters: a novel anticancer strategy? Nature Rev. Drug Discov. 2011, 10 (4), 261-275.
26. Neidle, S., Quadruplex Nucleic Acids as Novel Therapeutic Targets. J. Med. Chem. 2016, 59 (13), 5987-6011.
27. Davis, M. E.; Chen, Z. G.; Shin, D. M., Nanoparticle therapeutics: an emerging treatment modality for cancer. Nat. Rev. Drug Discov. 2008, 7 (9), 771-782.
28. Rosi, N. L.; Giljohann, D. A.; Thaxton, C. S.; Lytton-Jean, A. K. R.; Han, M. S.; Mirkin, C. A., Oligonucleotide-Modified Gold Nanoparticles for Intracellular Gene Regulation. Science 2006, 312 (5776), 1027-1030.
29. Whitehead, K. A.; Langer, R.; Anderson, D. G., Knocking down barriers: advances in siRNA delivery. Nat. Rev. Drug Discov. 2009, 8 (2), 129-138.
30. Tijerina, P.; Mohr, S.; Russell, R., DMS footprinting of structured RNAs and RNA-protein complexes. Nat. Protoc. 2007, 2 (10), 2608-2623.
31. Bhattacharyya, D.; Mirihana Arachchilage, G.; Basu, S., Metal Cations in G-Quadruplex Folding and Stability. Front. Chem. 2016, 4, 38, 1-14.
32. Kaczmarek, J. C.; Kowalski, P. S.; Anderson, D. G., Advances in the delivery of RNA therapeutics: from concept to clinical reality. Genome Med. 2017, 9 (1), 60.
33. Ranjan, S.; Sood, R.; Dudas, J.; Glueckert, R.; Schrott-Fischer, A.; Roy, S.; Pyykkö, I.; Kinnunen, P. K., Peptide-mediated targeting of liposomes to TrkB receptor-expressing cells. Int. J. Nanomedicine 2012, 7, 3475-3485.
34. Bien-Ly, N.; Yu, Y. J.; Bumbaca, D.; Elstrott, J.; Boswell, C. A.; Zhang, Y.; Luk, W.; Lu, Y.; Dennis, M. S.; Weimer, R. M.; Chung, I.; Watts, R. J., Transferrin receptor (TfR) trafficking determines brain uptake of TfR antibody affinity variants. J. Exp. Med. 2014, 211 (2), 233-244.
35. Porciani, D.; Signore, G.; Marchetti, L.; Mereghetti, P.; Nifosi, R.; Beltram, F., Two interconvertible folds modulate the activity of a DNA aptamer against transferrin receptor. Mol. Ther. Nucleic Acids 2014, 3 (1), e144.
36. Cheng, Y.; Meyers, J. D.; Agnes, R. S.; Doane, T. L.; Kenney, M. E.; Broome, P.; Burda, C.; Basilion, J. P., Addressing Brain Tumors with Targeted Gold Nanoparticles: A New Gold Standard for Hydrophobic Drug Delivery? Small 2011, 7 (16), 2301-2306.
37. Hoshyar, N.; Gray, S.; Han, H.; Bao, G., The effect of nanoparticle size on in vivo pharmacokinetics and cellular interaction. Nanomedicine-UK 2016, 11 (6), 673-692.
38. Porciani, D.; Cardwell, L. N.; Tawiah, K. D.; Alam, K. K.; Lange, M. J.; Daniels, M. A.; Burke, D. H., Modular cell-internalizing aptamer nanostructure enables targeted delivery of large functional RNAs in cancer cell lines. Nat. Commun. 2018, 9 (1), 2283-2283.
39. Foroozandeh, P.; Aziz, A. A., Insight into Cellular Uptake and Intracellular Trafficking of Nanoparticles. Nanoscale Res. Lett. 2018, 13 (1), 339.
40. Dauer, W.; Przedborski, S., Parkinson's Disease: Mechanisms and Models. Neuron 2003, 39 (6), 889-909.
41. Simola, N.; Morelli, M.; Carta, A. R., The 6-Hydroxydopamine model of parkinson's disease. Neurotox. Res. 2007, 11 (3), 151-167.
42. lacovitti, L.; Wei, X.; Cai, J.; Kostuk, E. W.; Lin, R.; Gorodinsky, A.; Roman, P.; Kusek, G.; Das, S. S.; Dufour, A.; Martinez, T. N.; Dave, K. D., The hTH-GFP reporter rat model for the study of Parkinson's disease. PloS one 2014, 9 (12), el13151.
43. Sun, D.; Thompson, B.; Cathers, B. E.; Salazar, M.; Kerwin, S. M.; Trent, J. O.; Jenkins, T. C.; Neidle, S.; Hurley, L. H., Inhibition of human telomerase by a G-quadruplex-interactive compound. J. Med. Chem. 1997, 40 (14), 2113-2116.
44. Xu, Y., Chemistry in human telomere biology: structure, function and targeting of telomere DNA/RNA. Chem. Soc. Rev 2011, 40 (5), 2719-2740.
45. Rodriguez, R.; Miller, K. M.; Forment, J. V.; Bradshaw, C. R.; Nikan, M.; Britton, S.; Oelschlaegel, T.; Xhemalce, B.; Balasubramanian, S.; Jackson, S. P., Small-molecule-induced DNA damage identifies alternative DNA structures in human genes. Nat. Chem. Biol. 2012, 8 (3). 301-310.
46. De Cian, A.; Lacroix, L.; Douarre, C.; Temime-Smaali, N.; Trentesaux, C.; Riou, J. F.; Mergny, J. L., Targeting telomeres and telomerase. Biochimie 2008, 90 (1), 131-155.
47. Schonhoft, J. D.; Bajracharya, R.; Dhakal, S.; Yu, Z.; Mao, H.; Basu, S., Direct experimental evidence for quadruplex-quadruplex interaction within the human ILPR. Nucleic Acids Res. 2009, 37 (10), 3310-3320.
48. Connor, A. C.; Frederick, K. A.; Morgan, E. J.; McGown, L. B., Insulin capture by an insulin-linked polymorphic region G-quadruplex DNA oligonucleotide. J. Am. Chem. Soc. 2006, 128 (15), 4986-4991.
49. Koukouraki, P.; Doxakis, E., Constitutive translation of human α-synuclein is mediated by the 5-untranslated region. Open Biol. 2016, 6 (4), 160022.
50. Simone, R.; Balendra, R.; Moens, T. G.; Preza, E.; Wilson, K. M.; Heslegrave, A.; Woodling, N. S.; Niccoli, T.; Gilbert-Jaramillo, J.; Abdelkarim, S.; Clayton, E. L.; Clarke, M.; Konrad, M.-T.; Nicoll, A. J.; Mitchell, J. S.; Calvo, A.; Chio, A.; Houlden, H.; Polke, J. M.; Ismail, M. A.; Stephens, C. E.; Vo, T.; Farahat, A. A.; Wilson, W. D.; Boykin, D. W.; Zetterberg, H.; Partridge, L.; Wray, S.; Parkinson, G.; Neidle, S.; Patani, R.; Fratta, P.; Isaacs, A. M., G-quadruplex-binding small molecules ameliorate C9orf72 FTD/ALS pathology in vitro and in vivo. EMBO Mol. Med. 2018, 10 (1), 22-31.
51. Zhou, W.; Brand, N. J.; Ying, L., G-quadruplexes-novel mediators of gene function. J. Cardiovasc. Transl. Res. 2011, 4 (3), 256-270.
52. Folini, M.; Venturini, L.; Cimino-Reale, G.; Zaffaroni, N., Telomeres as targets for anticancer therapies. Expert Opin. Ther. Targets 2011, 15 (5), 579-593.
53. Collie, G. W.; Parkinson, G. N., The application of DNA and RNA G-quadruplexes to therapeutic medicines. Chem. Soc. Rev. 2011, 40 (12), 5867-5892.
54. Tassinari, M., Zuffo, M., Pirota, V., Montalvo, A., Doria, F., Freccero, M., and Richter, S. Selective targeting of mutually exclusive DNA G-quadruplexes: HIV-1 LTR as paradigmatic model. Nucleic Acids Res. 2020, 48 (9), 4627-4642.
55. Mirihana Arachchilage, G.; Kharel, P.; Reid, J.; Basu, S., Targeting of G-Quadruplex Harboring Pre-miRNA 92b by LNA Rescues PTEN Expression in NSCL Cancer Cells. ACS Chem. Biol. 2018, 13 (4), 909-914.
56. Bhattacharyya, D.; Nguyen, K.; Basu, S., Rationally Induced RNA:DNA G-Quadruplex Structures Elicit an Anticancer Effect by Inhibiting Endogenous eIF-4E Expression. Biochemistry 2014, 53 (33), 5461-5470.
57. Xu, L.; Anchordoquy, T., Drug delivery trends in clinical trials and translational medicine: challenges and opportunities in the delivery of nucleic acid-based therapeutics. J. Pharm. Sci. 2011, 100 (1), 38-52.
58. Goldsmith, M.; Abramovitz, L.; Peer, D., Precision nanomedicine in neurodegenerative diseases. ACS nano 2014, 8 (3), 1958-1965.
59. Wilson, B.; Samanta, M. K.; Santhi, K.; Kumar, K. P.; Paramakrishnan, N.; Suresh, B., Poly(n-butylcyanoacrylate) nanoparticles coated with polysorbate 80 for the targeted delivery of rivastigmine into the brain to treat Alzheimer's disease. Brain Res. 2008, 1200, 159-168.
60. Wen, Z.; Yan, Z.; Hu, K.; Pang, Z.; Cheng, X.; Guo, L.; Zhang, Q.; Jiang, X.; Fang, L.; Lai, R., Odorranalectin-conjugated nanoparticles: preparation, brain delivery and pharmacodynamic study on Parkinson's disease following intranasal administration. J. Control. Release 2011, 151 (2), 131-138.
61. Helmschrodt, C.; Höbel, S.; Schöniger, S.; Bauer, A.; Bonicelli, J.; Gringmuth, M.; Fietz, S. A.; Aigner, A.; Richter, A.; Richter, F., Polyethylenimine Nanoparticle-Mediated siRNA Delivery to Reduce α-Synuclein Expression in a Model of Parkinson's Disease. Mol. Ther. Nucleic Acids 2017, 9, 57-68.
62. Ghosh, P.; Han, G.; De, M.; Kim, C. K.; Rotello, V. M., Gold nanoparticles in delivery applications. Adv. Drug Deliv. Rev. 2008, 60 (11), 1307-1315.
63. Zhou, J.; Rossi, J. J., Cell-Specific Aptamer-Mediated Targeted Drug Delivery. Oligonucleotides 2011, 21 (1), 1-10.
64. S F, D., Overcoming cellular barriers for RNA therapeutics. Nature Biotechnol. 2017, 35 (3), 222-229.
65. F R, M., Weak bases and ionophores rapidly and reversibly raise the pH of endocytic vesicles in cultured mouse fibroblasts. J. Cell Biol. 1982, 95 (2), 676-681.
66. K K, H.; H, P.; P H, S.; S A, W., A role for peptides in overcoming endosomal entrapment in siRNA delivery—A focus on melittin. Biotechnol. Adv. 2015, 33 (6), 931-940.
67. Pardridge, W. M., Tyrosine Hydroxylase Replacement in Experimental Parkinson's Disease with Transvascular Gene Therapy. NeuroRx 2005, 2 (1), 129-138.
68. Normalization of Striatal Tyrosine Hydroxylase and Reversal of Motor Impairment in Experimental Parkinsonism with Intravenous Nonviral Gene Therapy and a Brain-Specific Promoter. Hum. Gene Ther. 2004, 15 (4), 339-350.
69. Livak, K. J.; Schmittgen, T. D., Analysis of relative gene expression data using real-time quantitative PCR and the 2(-Delta Delta C(T)) Method. Methods 2001, 25 (4), 402-408.
70. Schmittgen, T. D.; Livak, K. J., Analyzing real-time PCR data by the comparative CT method. Nat. Protocols 2008, 3 (6), 1101-1108.
71. Beals, N.; Thiagarajan, P. S.; Soehnlen, E.; Das, A.; Reizes, O.; Lathia, J. D.; Basu, S., Five-Part Pentameric Nanocomplex Shows Improved Efficacy of Doxorubicin in CD44+ Cancer Cells. ACS Omega 2017, 2 (11). 7702-7713.

What is claimed is:

1. A method for controlling dopamine production in the treatment of neurological disorders, comprising the steps of:
a. providing an exogenous agent to regulate tyrosine hydroxylase (TH) expression in the catecholamine biogenesis pathway; and
b. incorporating the exogenous agent into a nanoparticle drug delivery system to deliver the agent to a dopaminergic neuron via targeting with a TrkB peptide aptamer, wherein the exogenous agent is a DNA-based molecule comprising an engineered G-quadruplex (GQ)-targeting DNA clip that is complementary to and targets a segment of the TH49 sequence of human tyrosine hydroxylase promoter.

2. The method according to claim 1, wherein the engineered G-quadruplex (GQ) targeting DNA Clip comprises a 5′GQ DNA Clip or a 3′GQ DNA Clip, and wherein the 5′GQ DNA Clip enhances tyrosine hydroxylase transcription and the 3′GQ DNA Clip represses tyrosine hydroxylase transcription.

3. The method according to claim 1, wherein the nanoparticle drug delivery system is a gold nanoparticle or a DNA nanoconjugate.

4. The method according to claim 2, wherein the engineered G-quadruplex (GQ)-targeting DNA clips block the formation of either the 5′GQ or the 3′GQ structures within the TH49 sequence of the human tyrosine hydroxylase promoter to favor the formation of one or the other of 5′GQ or 3′GQ structures.

5. An engineered G-quadruplex (GQ)-targeting DNA clip, complementary to a segment of TH49 sequence of human TH promoter, for modulating tyrosine hydroxylase expression, comprising: a 5′GQ DNA clip or a 3′GQ DNA clip in combination with a polyethylene glycol (PEG)-coated gold nanoparticle (AuNP) as a central tethering agent, PEGylated tyrosine receptor kinase B (TrkB), and transferrin receptor 1 (Tfr1) aptamers.

6. The method according to claim 1, further comprising the step of (c) administering the engineered G-quadruplex (GQ)-targeting DNA Clip to a human subject.

7. The method of claim 6, wherein the engineered G-quadruplex (GQ)-targeting DNA clip is incorporated into a nanoparticle delivery system prior to administration.

8. The method according to claim 7, wherein the nanoparticle drug delivery system is a gold nanoparticle or a DNA nanoconjugate.

9. The method according to claim 6, wherein the engineered G-quadruplex(GQ)-targeting DNA clip is a 5′GQ DNA clip or a 3′GQ DNA clip.

10. The method according to claim 1, wherein the neurological disorder is Parkinson's disease.

11. The method according to claim 6, wherein the neurological disorder is Parkinson's disease.

12. The method according to claim 1, wherein the neurological disorder is post-traumatic stress disorder (PTSD), schizophrenia, depression, drug addiction or attention deficient disorder (ADD).

13. The method according to claim 6, wherein the neurological disorder is post-traumatic stress disorder (PTSD), schizophrenia, depression, drug addiction or attention deficient disorder (ADD).

14. A 5′GQ Clip nanoparticle, comprising: a 5′GQ clip molecule, a polyethylene glycol (PEG)-coated gold nanoparticle (AuNP) as a central tethering agent, PEGylated tyrosine receptor kinase B (TrkB), and transferrin receptor 1 (Tfr1) aptamers.
